(19) United States
(12) Patent Application Publication
(10) Pub. No.: US 2025/0098994 A1
(43) Pub. Date: Mar. 27, 2025

(54) SILK-CHROMOPHORE COMPOSITE MATERIALS FOR IN SITU OXYGEN SENSING

(71) Applicants: Trustees of Tufts College, Medford, MA (US); Government of the United States as Represented by the Secretary of the Air Force, Wright-Patterson AFB, OH (US)

(72) Inventors: David L. Kaplan, Concord, MA (US); Thomas Falcucci, Boston, MA (US); Matthew Dalton, Bellbrook, OH (US); Kayla Presley, Dayton, OH (US); Tod Grusenmeyer, Dayton, OH (US); Jack Ly, Kettering, OH (US)

(21) Appl. No.: 18/823,853

(22) Filed: Sep. 4, 2024

Related U.S. Application Data
(63) Continuation of application No. PCT/US2023/014796, filed on Mar. 8, 2023.
(60) Provisional application No. 63/317,929, filed on Mar. 8, 2022.

(51) Int. Cl. A61B 5/1473; A61B 5/1459; A61K 49/00

(52) U.S. Cl. CPC A61B 5/14735; A61B 5/1459; A61K 49/0015; A61K 49/0073; A61B 2562/12

(57) ABSTRACT

Oxygen-sensing materials and methods are disclosed. The materials include oxygen-sensing chromophores embedded in a solid matrix including silk fibroin in an amount by weight of at least 50% of the total weight of the solid matrix. The solid matrix possesses at least partial transparency at relevant wavelengths. The solid matrix is biocompatible and biodegradable. The solid matrix is not a hydrogel. The chromophores are distributed throughout the solid matrix. Tissue oxygenation can be estimated from phosphorescence measurements made from oxygen-sensing materials implanted in subject.

### CROSS-REFERENCE TO RELATED APPLICATIONS

This application is a continuation of International Application Serial Number PCT/US2023/014796 (Attorney Docket No. 2095.0032), entitled “SILK-CHROMOPHORE COMPOSITE MATERIALS FOR IN SITU OXYGEN SENSING,” filed Mar. 8, 2023, and published as WO2023172612.

International Application Serial Number PCT/US2023/014796 claims priority to U.S. Provisional Patent Application No. 63/317,929 (Attorney Docket No. 2095.0031) filed Mar. 8, 2022, the entire contents of which are hereby incorporated by reference.

### STATEMENT REGARDING FEDERALLY SPONSORED RESEARCH

This invention was made with Government support from the U.S. Air Force. The Government of the United States has the right to practice or have practiced on behalf of the United States this subject invention throughout the world.

### FIELD

The disclosed technology is generally directed to an oxygen sensor. More particularly the technology is directed to an in vivo oxygen sensor.

### BACKGROUND

Physiological changes and the resultant alterations in the biochemical composition of the body reflect the overall health of an individual. In particular, soluble oxygen is a key indicator of physiological processes including exercise, sleep, stroke, and myocardial infarctions, among others. High-risk individuals who are susceptible to variations in ambient oxygen levels or undergo extreme fatigue include asthmatic patients, stroke patients, cardiac disease patients, as well as pilots or divers. Variations in the natural homeostatic oxygen levels provide insight into normal cycles as well as hazardous hypoxic and hyperoxic events, highlighting the utility of and need for real-time tissue oxygen sensors.

Many existing sensors function by sampling the composition of secreted fluids (i.e. sweat) or tissue components to detect the analyte of interest. However, biological systems are complex structures with heterogeneous compositions, architectures, and functions, which can often limit device sensitivity and selectivity for both wearable and implantable sensors. A Clark electrode implant has been the conventional method for monitoring oxygen concentrations in tissue. However, this type of sensor generally requires bulky and invasive equipment with readouts that are sensitive to electrical interference, and, therefore, has limited utility for biological applications. While there has been work towards translating this technology to wearable devices, microarrayed patches or sensor tattoos, the sensors are still limited by signal sensitivity and sampling selectivity.

In order to overcome these barriers, sensors are needed that integrate with native tissue without disrupting natural physiological processes and offer improved assessments of physiological functions in real time.

### SUMMARY

In an aspect, the present disclosure provides an oxygen-sensing material. The oxygen-sensing material includes a plurality of oxygen-sensing chromophores embedded in a solid matrix. The solid matrix includes silk fibroin in an amount by weight of at least 50% of the total weight of the solid matrix. The solid matrix is at least partially optically transparent to light at one or more wavelengths between 600 nm and 1300 nm. The solid matrix is biocompatible and biodegradable. The solid matrix is not a hydrogel. The plurality of oxygen-sensing chromophores are distributed throughout the solid matrix.

In another aspect, the present disclosure provides a method of making an oxygen-sensing material. The method comprises: a) dissolving silk fibroin and a plurality of oxygen-sensing chromophores into an organic solvent; b) removing the organic solvent, thereby resulting in the oxygen-sensing material comprising the plurality of oxygen-sensing chromophores embedded in a solid matrix comprising the silk fibroin. The amount of silk fibroin dissolved in step a) is selected to provide the solid matrix with silk fibroin in an amount by weight of at least 50% of the total weight of the solid matrix. The removing of step b) is tailored to provide the properties listed in the preceding paragraph.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1A. Schematic of a silk-chromophore composite implanted into subcutaneous tissue to continuously monitor tissue oxygenation.

FIG. 1B. Pictures of silk+Pd (II) tetramethacrylated benzoporphyrin (PdBMAP) film (left) and sponge (right).

FIG. 1C. Schematic of fabrication process for silk+PdBMAP sponge and film samples.

FIG. 2. Schematic of setup used to slow down the evaporation of the HFIP from silk films to ensure homogenous film topography and to prevent warping.

FIG. 3. Scanning electron microscopy (SEM) images of 5 and 10 w/v % silk films with and without PdBMAP.

FIG. 4. SEM images of silk sponges with and without PdBMAP.

FIG. 5A. Cell viability of human dermal fibroblast cells seeded onto silk and silk-PdBMAP films and sponges compared to results for tissue culture plastic (TCP).

FIG. 5B. Metabolic activity of human dermal fibroblast cells seeded onto silk and silk-PdBMAP films and sponges compared to results for tissue culture plastic (TCP).

FIG. 5C Mass loss of the silk-PdBMAP films and sponges aged in vitro in 37° C. phosphate-buffered saline (PBS) (black), PBS+1 U/mL α-chymotrypsin (blue), and PBS+1 U/mL protease XIV (red).

FIG. 6A Emission spectra (top) and excitation spectra (bottom) of PdBMAP-containing silk films and sponges in aerated 37° C. PBS.

FIG. 6B Phosphorescence decay curves (top) and resulting Stern-Volmer plot (bottom) of 5 w/v % silk-PdBMAP film in 37° C. PBS. The dotted line on the Stern-Volmer plots denotes the oxygen concentration at which the phosphorescence lifetime is quenched to 50% of its deaerated value.

FIG. 6C Phosphorescence decay curves (top) and resulting Stern-Volmer plot (bottom) of 10 w/v % silk-PdBMAP film in 37° C. PBS. The dotted line on the Stern-Volmer plots denotes the oxygen concentration at which the phosphorescence lifetime is quenched to 50% of its deaerated value.

FIG. 6D Phosphorescence decay curves (top) and resulting Stern-Volmer plot (bottom) of silk-PdBMAP sponge in 37° C. PBS. The dotted line on the Stern-Volmer plots denotes the oxygen concentration at which the phosphorescence lifetime is quenched to 50% of its deaerated value.

FIG. 7A. Monoexponential fits of phosphorescence decay curves for 5 w/v % silk+PdBMAP film at 0.5% (4.51 μM) dissolved oxygen in 37° C. PBS. IRF is shown in grey, and inset curve shows residuals resulting from a monoexponential reconvolution fit.

FIG. 7B. Monoexponential fits of phosphorescence decay curves for 5 w/v % silk+PdBMAP film at 1.0% O₂ (9.03 M) dissolved oxygen in 37° C. PBS. IRF is shown in grey, and inset curve shows residuals resulting from a monoexponential reconvolution fit.

FIG. 7C. Monoexponential fits of phosphorescence decay curves for 5 w/v % silk+PdBMAP film at 2.0% O₂ (18.1 μM) dissolved oxygen in 37° C. PBS. IRF is shown in grey, and inset curve shows residuals resulting from a monoexponential reconvolution fit.

FIG. 7D. Monoexponential fits of phosphorescence decay curves for 5 w/v % silk+PdBMAP film at 5.0% 02 (45.1 μM) dissolved oxygen in 37° C. PBS. IRF is shown in grey, and inset curve shows residuals resulting from a monoexponential reconvolution fit.

FIG. 7E. Monoexponential fits of phosphorescence decay curves for 5 w/v % silk+PdBMAP film at 10% O₂ (90.3 μM). IRF is shown in grey, and inset curve shows residuals resulting from a monoexponential reconvolution fit.

FIG. 8A. Biexponential fits of phosphorescence decay curves for 5 w/v % silk+PdBMAP film at 5.0% 02 (45.1 μM) dissolved oxygen concentrations in 37° C. PBS. IRF is shown in grey, and inset curve shows residuals resulting from a biexponential reconvolution fit.

FIG. 8B. Biexponential fits of phosphorescence decay curves for 5 w/v % silk+PdBMAP film at 10% O₂ (90.3 μM) dissolved oxygen concentration in 37° C. PBS. IRF is shown in grey, and inset curve shows residuals resulting from a biexponential reconvolution fit.

FIG. 9A. Monoexponential fits of phosphorescence decay curves for 10 w/v % silk+PdBMAP film at 0.5% O₂ (4.51 μM) dissolved oxygen concentration in 37° C. PBS. IRF is shown in grey, and inset curve shows residuals resulting from a monoexponential reconvolution fit.

FIG. 9B. Monoexponential fits of phosphorescence decay curves for 10 w/v % silk+PdBMAP film at 1.0% O₂ (9.03 μM) dissolved oxygen concentration in 37° C. PBS. IRF is shown in grey, and inset curve shows residuals resulting from a monoexponential reconvolution fit.

FIG. 9C. Monoexponential fits of phosphorescence decay curves for 10 w/v % silk+PdBMAP film at 2.0% O₂ (18.1 μM) dissolved oxygen concentration in 37° C. PBS. IRF is shown in grey, and inset curve shows residuals resulting from a monoexponential reconvolution fit.

FIG. 9D. Monoexponential fits of phosphorescence decay curves for 10 w/v % silk+PdBMAP film at 5.0% O₂ (45.1 μM) dissolved oxygen concentration in 37° C. PBS. IRF is shown in grey, and inset curve shows residuals resulting from a monoexponential reconvolution fit.

FIG. 9E. Monoexponential fits of phosphorescence decay curves for 10 w/v % silk+PdBMAP film at 10% O₂ (90.3 μM) dissolved oxygen concentration in 37° C. PBS. IRF is shown in grey, and inset curve shows residuals resulting from a monoexponential reconvolution fit.

FIG. 10A. Biexponential fits of phosphorescence decay curves for 10 w/v % silk+PdBMAP film at 5.0% O₂ (45.1 μM) dissolved oxygen concentration in 37° C. PBS. IRF is shown in grey, and inset curve shows residuals resulting from a biexponential reconvolution fit.

FIG. 10B. Biexponential fits of phosphorescence decay curves for 10 w/v % silk+PdBMAP film at 10% O₂ (90.3 μM) dissolved oxygen concentration in 37° C. PBS. IRF is shown in grey, and inset curve shows residuals resulting from a biexponential reconvolution fit.

FIG. 11A. Monoexponential fits of phosphorescence decay curves for silk+PdBMAP sponge at 0.5% O₂ (4.51 μM) dissolved oxygen concentration in 37° C. PBS. IRF is shown in grey, and inset curve shows residuals resulting from a monoexponential reconvolution fit.

FIG. 11B. Monoexponential fits of phosphorescence decay curves for silk+PdBMAP sponge at 1.0% O₂ (9.03 μM) dissolved oxygen concentration in 37° C. PBS. IRF is shown in grey, and inset curve shows residuals resulting from a monoexponential reconvolution fit.

FIG. 11C. Monoexponential fits of phosphorescence decay curves for silk+PdBMAP sponge at 2.0% O₂ (18.1 μM) dissolved oxygen concentration in 37° C. PBS. IRF is shown in grey, and inset curve shows residuals resulting from a monoexponential reconvolution fit.

FIG. 11D. Monoexponential fits of phosphorescence decay curves for silk+PdBMAP sponge at 5.0% O₂ (45.1 μM) dissolved oxygen concentration in 37° C. PBS. IRF is shown in grey, and inset curve shows residuals resulting from a monoexponential reconvolution fit.

FIG. 11E. Monoexponential fits of phosphorescence decay curves for silk+PdBMAP sponge at 10% O₂ (90.3 μM) dissolved oxygen concentration in 37° C. PBS. IRF is shown in grey, and inset curve shows residuals resulting from a monoexponential reconvolution fit.

FIG. 12A. Biexponential fits of phosphorescence decay curves for silk+PdBMAP sponge at 5.0% O₂ (45.1 μM) dissolved oxygen concentration in 37° C. PBS. IRF is shown in grey, and inset curve shows residuals resulting from a biexponential reconvolution fit

FIG. 12B. Biexponential fits of phosphorescence decay curves for silk+PdBMAP sponge at 10% O₂ (90.3 μM) dissolved oxygen concentration in 37° C. PBS. IRF is shown in grey, and inset curve shows residuals resulting from a biexponential reconvolution fit.

FIG. 13. Linear Stern-Volmer fit applied to silk-PdBMAP sponge. (R²=0.997)

FIG. 14A. Fits of unaged 10 w/v % silk+PdBMAP film for sample 1 at 3.0% O₂ (27.1 μM) dissolved oxygen concentration in 37° C. PBS. IRF is shown in grey, and inset curve shows residuals resulting from each reconvolution fit.

FIG. 14B. Fits of unaged 10 w/v % silk+PdBMAP film for sample 1 at 20.9% O₂ (189 μM) dissolved oxygen concentration in 37° C. PBS. IRF is shown in grey, and inset curve shows residuals resulting from each reconvolution fit.

FIG. 14C. Fits of unaged 10 w/v % silk+PdBMAP film for sample 2 at 3.0% 02 (27.1 μM) dissolved oxygen concentration in 37° C. PBS. IRF is shown in grey, and inset curve shows residuals resulting from each reconvolution fit.

FIG. 14D. Fits of unaged 10 w/v % silk+PdBMAP film for sample 2 at 20.9% O₂ (189 μM) dissolved oxygen concentration in 37° C. PBS. IRF is shown in grey, and inset curve shows residuals resulting from each reconvolution fit.

FIG. 15A. Fits of unaged silk+PdBMAP sponge for sample 1 at 3.0% 02 (27.1 μM) dissolved oxygen concentration in 37° C. PBS. IRF is shown in grey, and inset curve shows residuals resulting from each reconvolution fit.

FIG. 15B. Fits of unaged silk+PdBMAP sponge for sample 1 at 20.9% 02 (189 μM) dissolved oxygen concentration in 37° C. PBS. IRF is shown in grey, and inset curve shows residuals resulting from each reconvolution fit.

FIG. 15C. Fits of unaged silk+PdBMAP sponge for sample 2 at 3.0% 02 (27.1 μM) dissolved oxygen concentration in 37° C. PBS. IRF is shown in grey, and inset curve shows residuals resulting from each reconvolution fit.

FIG. 15D. Fits of unaged silk+PdBMAP sponge for sample 2 at 20.9% 02 (189 μM) dissolved oxygen concentration in 37° C. PBS. IRF is shown in grey, and inset curve shows residuals resulting from each reconvolution fit.

FIG. 16A. Fits of 10 w/v % silk+PdBMAP film for sample 1 at 3.0% 02 (27.1 μM) dissolved oxygen concentration in 37° C. PBS after aging in 37° C. PBS+1 U/mL α-chymotrypsin for 4 days. IRF is shown in grey, and inset curve shows residuals resulting from each reconvolution fit.

FIG. 16B. Fits of 10 w/v % silk+PdBMAP film for sample 1 at 20.9% 02 (189 μM) dissolved oxygen concentration in 37° C. PBS after aging in 37° C. PBS+1 U/mL α-chymotrypsin for 4 days. IRF is shown in grey, and inset curve shows residuals resulting from each reconvolution fit.

FIG. 16C. Fits of 10 w/v % silk+PdBMAP film for sample 2 at 3.0% 02 (27.1 μM) dissolved oxygen concentration in 37° C. PBS after aging in 37° C. PBS+1 U/mL α-chymotrypsin for 4 days. IRF is shown in grey, and inset curve shows residuals resulting from each reconvolution fit.

FIG. 16D. Fits of 10 w/v % silk+PdBMAP film for sample 2 at 20.9% 02 (189 μM) dissolved oxygen concentration in 37° C. PBS after aging in 37° C. PBS+1 U/mL α-chymotrypsin for 4 days. IRF is shown in grey, and inset curve shows residuals resulting from each reconvolution fit.

FIG. 17A. Fits of silk+PdBMAP sponge for sample 1 at 3.0% 02 (27.1 μM) dissolved oxygen concentration in 37° C. PBS after aging in 37° C. PBS+1 U/mL α-chymotrypsin for 4 days. IRF is shown in grey, and inset curve shows residuals resulting from each reconvolution fit.

FIG. 17B. sample 1, Fits of silk+PdBMAP sponge for sample 1 at 20.9% 02 (189 μM) dissolved oxygen concentration in 37° C. PBS after aging in 37° C. PBS+1 U/mL α-chymotrypsin for 4 days. IRF is shown in grey, and inset curve shows residuals resulting from each reconvolution fit.

FIG. 17C. Fits of silk+PdBMAP sponge for sample 2 at 3.0% 02 (27.1 μM) dissolved oxygen concentration in 37° C. PBS after aging in 37° C. PBS+1 U/mL α-chymotrypsin for 4 days. IRF is shown in grey, and inset curve shows residuals resulting from each reconvolution fit.

FIG. 17D. sample 2, Fits of silk+PdBMAP sponge for sample 1 at 20.9% 02 (189 μM) dissolved oxygen concentration in 37° C. PBS after aging in 37° C. PBS+1 U/mL α-chymotrypsin for 4 days. IRF is shown in grey, and inset curve shows residuals resulting from each reconvolution fit.

FIG. 18. Lifetimes in 37° C. PBS at 3.0% 02 (27.1 μM) and 20.9% 02 (189 μM) of silk-PdBMAP 10 w/v % silk film and sponge before and after aging in 37° C. PBS+1 U/mL α-chymotrypsin for 4 days.

FIG. 19. Normalized emission spectra in aerated 37° C. PBS of silk-PdBMAP 10 w/v % silk film and sponge before and after aging in 37° C. PBS+1 U/mL α-chymotrypsin for 4 days.

FIG. 20. Normalized excitation spectra in aerated 37° C. PBS of silk-PdBMAP 10 w/v % silk film and sponge before and after aging in 37° C. PBS+1 U/mL α-chymotrypsin for 4 days.

FIG. 21A. Schematics of example implant locations for film and sponge samples (left), testing setup for measurements with rat wearing a nose cone supplying anesthesia/O₂ providing a hyperoxic condition (right), and testing schedule for in vivo experiments (bottom).

FIG. 21B. Hematoxylin and eosin staining of sponge and film samples at days 7 and 28 (scale bar=1,000 μm).

FIG. 21C. Lifetime versus time results for select nose cone removal experiment wherein nose cone providing anesthesia/O₂ was removed, allowing the rat to breathe in ambient oxygen before the nose cone was readministered after approximately one minute. Results shown are for day 18. In vitro results of the lifetime of the unaged sensor at various oxygen concentrations are shown for comparison.

FIG. 21D. Lifetime over the course of days 14-28 for all samples. On each day, results are shown for the lifetimes observed when the rat was under hyperoxia (with nose cone providing anesthesia/O₂) and normoxia (without nose cone). In vitro results of the lifetime of the unaged sensor at various oxygen concentrations are shown for comparison.

FIG. 22. Histological images of 10 w/v % silk films with and without PdBMAP after 7, 14, and 28 days (scale bar=1,000 μm).

FIG. 23. Histological images of silk sponges with and without PdBMAP after 7, 14, and 28 days (scale bar=1,000 μm).

FIG. 24. Lifetime versus time results for nose cone removal experiments for silk-PdBMAP film implanted in rat 1. A nose cone supplying anesthesia/O₂ (hyperoxic condition) was removed, allowing the animal to breathe ambient oxygen (normoxic condition) until the nose cone was reapplied after approximately one minute. Results shown are for days: A) 14, B) 18, C) 21, D) 25, and E) 28.

FIG. 25. Lifetime versus time results for nose cone removal experiments for silk-PdBMAP sponge implanted in rat 1. A nose cone supplying anesthesia/O₂ (hyperoxic condition) was removed, allowing the animal to breathe ambient oxygen (normoxic condition) until the nose cone was reapplied after approximately one minute. Results shown are for days: A) 14, B) 18, C) 21, D) 25, and E) 28.

FIG. 26. Lifetime versus time results for nose cone removal experiments for silk-PdBMAP film implanted in rat 2. A nose cone supplying anesthesia/O₂ (hyperoxic condition) was removed, allowing the animal to breathe ambient oxygen (normoxic condition) until the nose cone was reapplied after approximately one minute. Results shown are for days: A) 14, B) 18, C) 21, D) 25, and E) 28.

FIG. 27. Lifetime versus time results for nose cone removal experiments for silk-PdBMAP sponge implanted in rat 2. A nose cone supplying anesthesia/O₂ (hyperoxic condition) was removed, allowing the animal to breathe ambient oxygen (normoxic condition) until the nose cone was reapplied after approximately one minute. Results shown are for days: A) 14, B) 18, C) 21, D) 25, and E) 28.

FIG. 28. Lifetime versus time as rat originally breathed in ambient oxygen and was then subjected to CO₂ during the standard euthanasia process (left) and example decay curves along with the monoexponential fits of their lifetimes shown in red (right) for A) 10 w/v % silk-PdBMAP film implanted in Rat 2 and B) Sponge-PdBMAP implanted in Rat 1. In vitro results of the lifetime of the unaged sensor at various oxygen concentrations are shown for comparison.

### DETAILED DESCRIPTION OF THE INVENTION

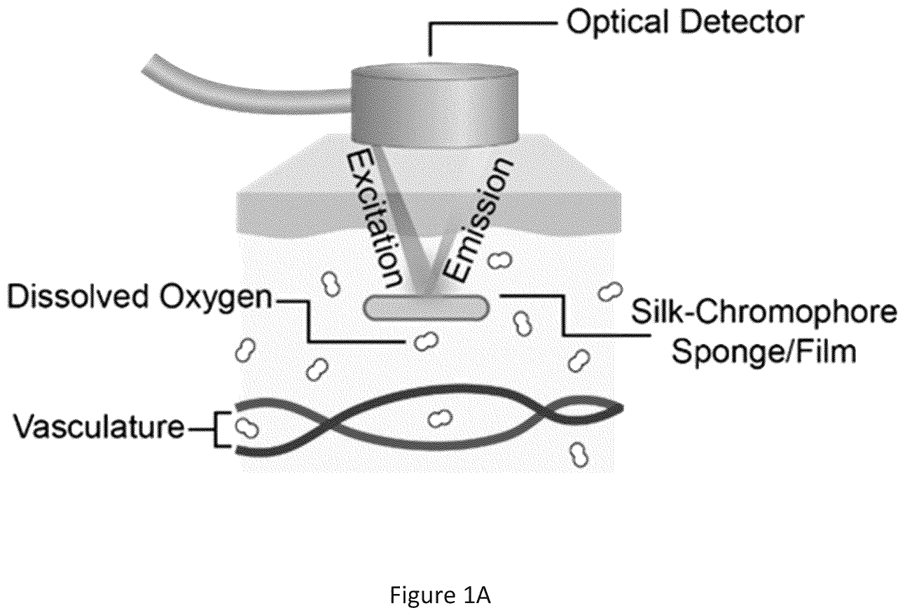

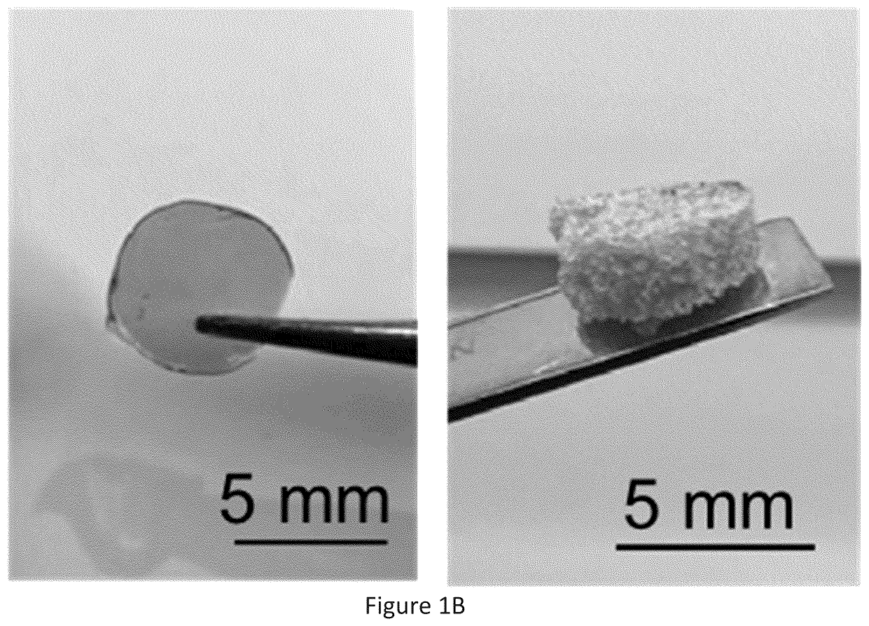

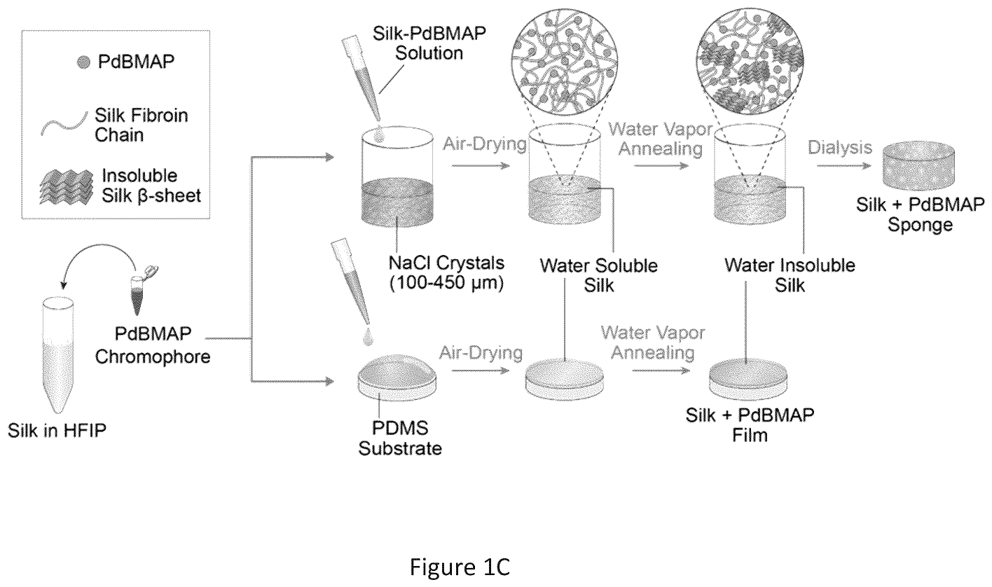

Medical conditions and/or athletic performance can benefit from continuous monitoring of tissue oxygenation in vivo.[1,2] Implantable and insertable sensors can be used to measure and report tissue oxygenation levels, which support effective treatment of numerous medical conditions[3], including cancer[4], critical limb ischemia[5], metabolic diseases[6], sepsis[7], and pulmonary diseases[8], including the life-threatening “silent hypoxia” that can result from COVID-19 pneumonia.[9] Along with an external reader, an implantable optical oxygen sensor can monitor tissue oxygenation while avoiding the implantation of any electronics into the body. The implantable oxygen sensor can include an oxygen-sensing material. Generally, the oxygen-sensing material can include an oxygen-sensing chromophore incorporated within a matrix, such as a silk-based sponge or film. The optical properties of the oxygen-sensing chromophore in the matrix are affected by interactions with oxygen in the surrounding tissue environment. The matrix can be biocompatible such that the oxygen-sensing material can be implanted in living tissue as shown in FIG. 1A.

### Oxygen-Sensing Chromophore

The oxygen-sensing chromophore can be a molecule having absorption and emission wavelength maxima within the optical tissue window, which is defined as between 600 nm and 1300 nm. In the optical tissue window, light penetration depth is appropriate for sensor use due to reduced Rayleigh and Mie scattering and absorption of light by the biological pigments (e.g., hemoglobin and melanin) and water being relatively low as compared to absorption of light in other ranges of wavelengths.[25,26] The oxygen-sensing chromophore can exhibit a long-lived phosphorescence lifetime. In the absence of oxygen, the phosphorescence emission of the oxygen-sensing chromophore is the most intense. When oxygen is present, for example as dissolved oxygen in tissue, the oxygen quenches the intensity of the emission of the oxygen-sensing chromophore. Furthermore, the emission lifetime of the oxygen-sensing chromophore is longer in the absence of oxygen. The presence of oxygen decreases the emission lifetime of the oxygen-sensing chromophore.

Generally, the oxygen-sensing chromophore can be a molecule having an extended π-system and having oxygen-sensitive absorption and emission wavelength maxima within the optical tissue window. In one example, a phosphorescent benzoporphyrin can be used as the oxygen-sensing chromophore. Benzoporphyrins have a Q-band absorption and emission wavelength maximum within the optical tissue window.[25] For example, Pd (II) tetramethacrylated benzoporphyrin (PdBMAP), is a chromophore that operates in the optical tissue window.[11,28,29] In other examples, the oxygen-sensing chromophore can be Pt(II) benzoporphyrins, Pd(II) benzoporphyrins, or any combination thereof. The oxygen-sensing chromophore can include substituted benzoporphyrins, such as tetraphenyltetrabenzoporphyrin. In another example, a naphthoporphyrin can be used as the oxygen-sensing chromophore. Boron-dipyrromethene based chromophores with extended π-systems that enable absorption and emission within the optical tissue window can also be utilized with similar transition metals (i.e. palladium, platinum, rhenium, and gold) or halides (i.e. iodine) which induce oxygen-sensitive, phosphorescence emission.

According to an aspect as disclosed herein, the oxygen-sensing chromophore can be soluble in organic solvents such as alkanes (e.g., pentane, hexane, heptane), aromatics (e.g., benzene, toluene, xylene), acetic acid, diethyl ether, ethyl acetate, chloroform, methylene chloride, pyridine, DMSO or 1,1,1,3,3,3-hexafluoro-2-propanol (HFIP). The oxygen-sensing chromophore can have a low water solubility. For example, the solubility of the oxygen-sensing chromophore in water at pH 7.4 and 37° C. can be less than 0.0001 mg/mL, less than 0.00025 mg/mL, less than 0.0005 mg/mL, less than 0.00075 mg/mL, less than 0.001 mg/mL, less than 0.0025 mg/mL, less than 0.005 mg/mL, less than 0.0075 mg/mL, or less than 0.01 mg/mL.

### Matrix

The matrix for the oxygen-sensing material can include a silk protein-based material. Silk protein-based materials are biocompatible as implants and have been shown to integrate well with surrounding tissues. Silk-protein based materials are optically transparent to visible light. More specifically, silk-protein based materials do not absorb within the optical tissue window. Silk-protein based materials can be bioresorbable. For example, silk-based protein materials can be at least partially enzymatically degradable. The silk-based protein material can include silk fibroin.

The matrix can include silk fibroin in a solid form. The matrix can include silk fibroin in an amount by weight of at least 55%, at least 60%, at least 65%, at least 70%, at least 75%, at least 80%, at least 85%, at least 90%, at least 95%, at least 99%, or 99.75% of the total weight of the solid matrix. In one example, at least 50% of the silk fibroin has a molecular weight of at least 60 kDa, at least 70 kDa, at least 80 kDa, at least 90 kDa, at least 100 kDa, at least 110 kDa, at least 120 kDa, at least 130 kDa, or at least 140 kDa. In another example, the silk fibroin has a number average molecular weight of at least 80 kDa, at least 90 kDa, at least 100 kDa, at least 110 kDa, at least 120 kDa, at least 130 kDa, at least 140 kDa, at least 150 kDa, or at least 160 kDa, and the number average molecular weight is at most 400 kDa.

Silk-based protein materials can be manipulated to form the matrix in a variety of solid material formats. For example, the material formats can be porous, such as a foam or a sponge. In another example, the material format can be a film. In yet another example, the material format is not a hydrogel.

### Oxygen-Sensing Material

The oxygen-sensing material can include a plurality of oxygen-sensing chromophores embedded or encapsulated within the matrix. The oxygen-sensing material can be formed in a porous material format. For example, the material format can include pores in the range of 100-450 μm in order to achieve interconnected pores large enough to promote cellular infiltration and tissue integration. The oxygen-sensing material can demonstrate a measurable resistance to compression. For example, the oxygen-sensing material can have a compressive modulus of at least 1 kPa, at least 10 kPa, at least 20 kPa, at least 30 kPa, at least 40 kPa, at least 50 kPa, at least 60 kPa, at least 70 kPa, at least 80 kPa, at least 90 kPa, or at least 100 kPa. In another example, the oxygen-sensing material can have a tensile modulus of at least 1 MPa, at least 50 MPa, at least 100 MPa, at least 150 MPa, at least 200 MPa, at least 250 MPa, at least 300 MPa, at least 350 MPa, or at least 500 MPa.

The oxygen-sensing material can be cytocompatible with human cells. For example, the oxygen-sensing material can host dermal fibroblast cells on the surface or within the pores. These cells can readily spread onto the silk substrates. Furthermore, the oxygen-sensing material can be biodegradable with a tunable dissolution rate. For example, in pure water at 37° C. the dissolution rate can be at least 70% of initial mass remaining after 2 weeks. Additionally and alternatively, the dissolution rate can be at least 75% of initial mass, at least 80% of initial mass, at least 85% of initial mass, at least 90% of initial mass, at least 95% of initial mass, or 100% of initial mass remaining after 2 weeks.

### A Method of Making an Oxygen-Sensing Material

Silk protein-based materials can be processed through an established water-based method employing salts to dissolve the protein producing an aqueous silk solution. The oxygen-sensing chromophore can be incorporated into the aqueous solution using an aqueous miscible organic solvent. The resultant silk-chromophore solution can then be used to produce solid material formats such as films, gels, or the like.

Silk protein-based materials can be processed through an organic solvent-based method. In this method, silk protein is dissolved in a volatile organic solvent along with the oxygen-sensing chromophore as a powder to produce a silk-chromophore organic solution. This solution can then be further used to produce solid material formats such as sponges with interconnected porosity, films with varied thickness, and the like, by exploiting the high volatility of the organic solvent.

More specifically, the matrix can be generated by preparing a first solution including the silk protein-based material, such as silk fibroin, in an organic solvent, such as HFIP. The first solution can be between 1 w/v % and 15 w/v %. A second solution can be prepared to include the oxygen-sensing chromophore dissolved in an organic solvent, such as HFIP. The second solution can include at least 10 mg/mL of the oxygen-sensing chromophore. The first solution of the silk protein-based material can be mixed with the second solution in amounts appropriate to form a matrix solution having a concentration of between 0.05 w/w % and 0.25 w/w % of the oxygen-sensing chromophore relative to the mass of the silk protein-based material. The solvent can be removed from the matrix solution such that the matrix solution can form a film or a sponge according to processes previously described. (See Example 1) The matrix can include at least 50%, at least 60%, at least 70%, at least 80%, at least 90%, at least 99%, or at least 99.75% silk fibroin by weight of the total matrix. The oxygen-sensing chromophores are mixed with the silk protein-based material in the matrix solution; thus the oxygen-sensing chromophores are distributed throughout the resultant oxygen-sensing material. In some cases, the chromophores are distributed homogeneously. In some cases, the chromophores are distributed in a desired concentration profile, which can be non-homogeneous.

### A Method of Using an Oxygen-Sensing Material:

In the absence of oxygen, phosphorescence emission of the oxygen-sensing chromophore has the highest intensity and exhibits the longest lifetime. The deaerated phosphorescence lifetime (T₀) is the phosphorescence lifetime in the absence or substantial absence of oxygen. When oxygen contacts the oxygen-sensing chromophore, both the phosphorescence intensity (I) and lifetime (τ) are quenched. Thus, it is possible to use either a ratiometric intensity or a lifetime-based detection to determine the amount of oxygen present using the oxygen-sensing material.

The Stern Volmer equation relates the oxygen concentration to the phosphorescence intensity (I) and lifetime (τ) of the oxygen-sensing chromophore. The sensitivity is described by the Stern-Volmer quenching rate constant (K_{SV}), which equals the product of the deaerated lifetime (T₀) and the bimolecular quenching rate constant (k_{q}) (Eq. 2). If the oxygen-sensing chromophore is in solution or is homogenously distributed in a matrix, a Stern Volmer plot (Eq. 1)[13] is linear and the decay can be monoexponential.

In one example, the phosphorescence lifetime decay at a given dissolved oxygen concentration can be obtained by exciting the oxygen-sensing chromophore at a specific excitation wavelength (e.g., λ_{Ex}: 630 nm) using a light source. The emission can be then monitored at the maximum emission wavelength (for example, λ_{Em}: 804-805 nm) to obtain a decay curve. The decay curve can be fit to either a monoexponential (Eq. 4) and/or biexponential (Eq. 5) equation. The fit can provide the phosphorescence lifetime.

The deaerated phosphorescence lifetime can be measured at substantially zero soluble oxygen concentration or obtained from a predictive method as described elsewhere [16]. The oxygen-sensing material can provide the phosphorescence lifetime that is 50% of the deaerated phosphorescence lifetime at a soluble oxygen concentration of between 5 μM and 50 μM, including at least 5 μM, at least 10 μM, at least 15 μM, at least 20 μM, at least 25 μM, at least 30 μM, at least 35 μM, at least 40 μM, or at least 45 μM, and at most 50 μM, at most 45 μM, at most 40 μM, at most 35 μM, at most 30 μM, at most 25 μM, at most 20 μM, at most 15 μM, or at most 10 μM. A phosphorescence decay curve measured from the oxygen-sensing material can have a biexponential fit resulting in an amplitude average lifetime that varies from the lifetime resulting from a monoexponential fit of the phosphorescence decay curve by at most 10%, at most 8%, at most 6%, at most 4%, at most 2%, or substantially 0%.

The oxygen-sensing material can be used in vivo, for example as an implant, to acquire phosphorescence data corresponding to the concentration of oxygen in living tissue. The implantable oxygen sensor material can be the oxygen-sensing material as described herein, for example in the form of a film or a sponge. The implantable oxygen sensor material can be implanted within a mammalian organism, for example the implantable oxygen sensor material can be implanted subcutaneously. The response of the implantable oxygen sensor material to oxygen levels in the surrounding tissue can be monitored externally using a light source and detector. The integration of the implantable oxygen sensor material into the living tissue can include formation of vascular structures within the pores of the implantable oxygen sensor material. Such integration can improve diffusion and oxygen transfer to the oxygen-sensing chromophore within the implantable oxygen sensor material, thus improving the response time of the implantable oxygen sensor material.

The ability to continuously monitor tissue oxygenation in vivo is greatly desired for a variety of medical applications. Bioresorbable, implantable optical oxygen sensors as disclosed herein can be formed from silk-based matrices and oxygen-sensitive chromophores. Silk provides a beneficial matrix material due to its biocompatibility, its ability to impart a stabilizing effect on incorporated additives due to its amphiphilic nature and the potential to tune the degradation of the silk matrix without having to alter the chemical composition. A phosphorescent chromophore can be incorporated in the silk matrix to impart oxygen sensitivity. According to this disclosure, implantable optical oxygen sensors can be produced using an organic solvent-based processing method to combine a phosphorescent chromophore with a silk matrix, where silk-chromophore films with varied thickness or silk-chromophore sponges with interconnected porosity are fabricated. The implantable optical oxygen sensors can integrate well with surrounding tissues after implantation in a living organism and can respond quickly to oxygen challenges. The implantable optical oxygen sensors as disclosed herein demonstrate high biocompatibility and exhibit excellent photophysical properties with oxygen sensitivities (i.e., Stern-Volmer quenching rate constants of 2.7-3.2×10⁴ M⁻¹) that are ideal for both monitoring physiological tissue oxygen levels and for detecting deviations from normal behavior (e.g., hyperoxia, hypoxia). The implantable optical oxygen sensors can degrade over time while maintaining oxygen sensing capability. The implantable optical oxygen sensors can consistently monitor tissue oxygenation in vivo as demonstrated by a multi-week rodent study.

### EXAMPLES

### Example 1

### Introduction

To monitor medical conditions and/or athletic performance, implantable and insertable sensors are desired for real-time, continuous monitoring.[1,2] Tissue oxygenation is especially critical for numerous conditions[3], such as various cancers[4], critical limb ischemia[5], metabolic diseases[6], sepsis[7], and pulmonary diseases[8], including the life-threatening “silent hypoxia” that can result from COVID-19 pneumonia.[9] Along with an external reader, an implantable optical oxygen sensor can monitor tissue oxygenation while avoiding the implantation of any electronics into the body. Implantable oxygen sensors sample the surrounding tissue environment to directly measure tissue oxygenation, a parameter that is related to but distinct from SpO₂ obtained from pulse oximetry (i.e., percent of hemoglobin bound to oxygen). In fact, changes in tissue oxygenation were found to be more drastic than SpO₂ changes in response to applied stimuli.[11] Tissue oxygen sensors also allow for detection of both hypoxia (i.e., low tissue O₂) and hyperoxia (i.e., high tissue O₂), while pulse oximetry is not well equipped to measure hyperoxia.

Optical oxygen sensors typically involve the incorporation of a chromophore with a long-lived, phosphorescence lifetime into a solid matrix. In the absence of oxygen, phosphorescence emission is the most intense and exhibits the longest lifetime, while dissolved oxygen quenches intensity and lifetime in the same manner via a collisional process.[13] For real-world applications, it is preferable to use either ratiometric intensity- or lifetime-based detection, since intensity measurements are affected by excitation light brightness, the exact position of the detector and sensor, ambient light, and other factors.[13,14]

The matrix material and the method by which the chromophore is incorporated can significantly impact oxygen-sensitivity and applicability for real-time monitoring.[13,15,16] Generally, a chromophore that is in solution or homogeneously distributed in a matrix results in a linear Stern Volmer plot (Eq. 1)[13] whereby oxygen concentration is inversely proportional to the phosphorescence intensity (I) and lifetime (τ). The sensitivity is described by the Stern-Volmer quenching rate constant (K_{SV}), which equals the product of the deaerated lifetime (T₀) and the bimolecular quenching rate constant (k_{q}) (Eq. 2). Linear Stern-Volmer behavior is often accompanied by monoexponential decays with both attributes signifying a homogenous chromophore environment.

$${I}_{0} / I = {T}_{0} / T = I + {K}_{SV} [ {O}_{2} ] \qquad ( 1 )$$

$${K}_{SV} = {T}_{0} {k}_{q} \qquad ( 2 )$$

However, in solid matrices, the chromophore environment is often heterogeneous resulting in multi-exponential decays and Stern-Volmer plots with a downward curve.[17] Numerous theories are used to describe this behavior[18-22], and while the physical basis varies, multiple models may fit the data in a mathematically equivalent manner.[18] The pervasive multi-site model, often simplified to the two-site model[23], attributes this behavior to the existence of multiple populations of chromophores that exhibit different K_{SV} values (Eq. 3)[18] Here, fractional contributions of each site are designated as f_{0,1} and f_{0,2}, where f_{0,2}=1−f_{0,1}.

$${I}_{0} / I = {T}_{0} / {T}_{m} = {[ ( {f}_{0 , 1} / ( 1 + {K}_{SV , 1} [ {O}_{2} ] ) ) + ( {f}_{0 , 2} / ( 1 + {K}_{SV , 2} [ {O}_{2} ] ) ) ]}^{- 1} \qquad ( 3 )$$

The ability to quickly interpret the optical measurements is especially important for real-time continuous monitoring and will be easiest when the chromophore environment is sufficiently homogeneous such that a linear Stern-Volmer plot and/or monoexponential decays can describe or approximate its behavior. Both the matrix material and processing conditions can impact this ability as a heterogeneous chromophore environment could result from different domains in the matrix and/or chromophore aggregation.

There are additional challenges associated with the task of designing implantable oxygen sensors. Matrix materials must be biocompatible and avoid adverse foreign-body immune responses.[24] In addition, the excitation and emission wavelengths for many oxygen-sensitive chromophores (e.g., Pd (II) and Pt (II) porphyrins, Ru (II) polypyridyl complexes) have poor penetration depths due to the high degrees of absorption and/or scattering in tissues.[14,25,26] However, annulating pyrrole rings with benzo-adducts allow for the generation of benzoporphyrins that are significantly red-shifted compared to their non-benzo counterparts.[27] From this modification, the Q-band absorption and emission wavelength maximum can be within the optical tissue window (600-1300 nm).[25] This window is the region with optimal penetration depth due to reduced Rayleigh and Mie scattering[26] and relatively low absorption of the major native pigments (e.g., hemoglobin and melanin) and water.[25,26]

Oxygen-sensing platforms have been developed to overcomes these challenges, such as with a porous, tissue-integrating poly(2-hydroxyethyl methacrylate) (polyHEMA) hydrogel and Pd (II) tetramethacrylated benzoporphyrin (PdBMAP), a chromophore that operates in the optical tissue window. This oxygen-sensing platform can be injected into tissue, and its phosphorescence lifetime can be monitored with an external reader.[10, 11,28] However, a bioresorbable alternative would enhance user acceptance of such technology, particularly if the degradation time could be tailored according to the application or user preference. Previous work on bioresorbable, dissolved oxygen sensors is limited, and includes PdBMAP-containing electrospun synthetic/natural polymer blends for implantable applications[16,29] and boron dye-containing polylactide films for wound dressings.[30-32]

However, use of natural polymers could enhance implant tissue integration as the system would be susceptible to remodeling processes in tissue. Silk fibroin is a unique and versatile material for such an application as it is biocompatible, biodegradable due to proteases in vivo, highly versatile in terms of material formats with robust properties, and is non-inflammatory.[33] It can be processed into a variety of formats (e.g., sponges, films, electrospun fibers, hydrogels) through both aqueous and non-aqueous methods.[34] Silk offers tunable degradation rate[33] and excellent mechanical properties compared to other natural polymers.[35] Additionally, the various hydrophobic and hydrophilic domains along the protein drive polymer self-assembly and impart a stabilizing effect on additives or dopants (i.e., the incorporated chromophore in the case of optical sensors).[36,37]

The fabrication of aqueous-processed silk-PdBMAP oxygen-sensing films has previously been investigated, where the degradation time could be tuned without compositional changes.[38] However, films are not an ideal form factor as their non-porous nature limits remodeling to the surface or interface, inhibiting effective tissue integration. Additionally, while the aqueous-based processing (i.e., DI H₂O with <1% dimethyl sulfoxide (DMSO)) could be advantageous for biocompatibility reasons, careful control over environmental conditions was necessary to prevent aggregation of the added water-insoluble Pd (II) benzoporphyrin. Nevertheless, the chromophore environment was still heterogeneous as evidenced by biexponential phosphorescence decays and the downward-curving Stern-Volmer plots.[38]

In order to overcome these barriers, there is a need for sensors that integrate with native tissue without disrupting natural physiological processes, sensors that are resorbable, and sensors that offer improved assessments of physiological functions in real time. The sensors should be inexpensive, minimally invasive, and not susceptible to background noise.

In the present disclosure, versatile silk protein-based material formats for oxygen-sensing are disclosed. These silk-based material formats demonstrate bioresorbable, implantable optical oxygen sensors that can integrate with the surrounding tissues. Silk was chosen as the matrix material due to its unique chemistry to interact with chromophores, biocompatibility, and the potential to tune degradation lifetime without altering chemical composition. A phosphorescent Pd (II) benzoporphyrin chromophore was incorporated to impart oxygen-sensitivity. Organic solvent-based processing methods using 1,1,1,3,3,3-hexafluoro-2-propanol were used to fabricate: 1) silk-PdBMAP films with varied thickness and 2) silk-PdBMAP sponges with interconnected porosity. All compositions were biocompatible and exhibited photophysical properties with oxygen-sensitivities (i.e., Stern-Volmer quenching rate constants of 2.7-3.2×10⁴ M⁻¹) useful for monitoring physiological tissue oxygen levels and for detecting deviations from normal behavior (e.g., hyperoxia).

The examples included herein demonstrate the tuning of degradation time without significantly impacting photophysical properties. Furthermore, the ability to consistently monitor tissue oxygenation in vivo using the sensors as disclosed herein has been established via a multi-week rodent study. The sponges as disclosed herein have been successfully integrated into tissue, according to histological assessments. Further, the examples demonstrate that the material format of sponge responds more quickly to various oxygen challenges than the material format of films.

### Materials

Bombyx mori silk cocoons were obtained from Tajima Shoji Co., Ltd (Yokohama, Japan). HFIP, NaCO₃, LiBr, sucrose, isopentane, hematoxylin and eosin (H&E) stains, protease XIV from Streptomyces griseus, and α-chymotrypsin from bovine pancreas were purchased from Sigma-Aldrich (Sigma-Aldrich, St. Louis, MO, USA). Sylgard 184 elastomer kit polydimethylsiloxane (PDMS) was acquired from Dow (Midland, MI, USA). Dulbecco's phosphate-buffered saline (PBS), Dulbecco's modified Eagle's medium (DMEM), high-glucose Glutamax, AlamarBlue, Live/Dead viability kit, antibiotic-antimycotic, fetal bovine serum (FBS), 4% buffered paraformaldehyde, Tissue Path Superfrost Plus gold slides, and optimal cutting temperature (OCT) compound were purchased from Thermo Fisher Scientific (Waltham, MA, USA). Primary human dermal fibroblasts were obtained from ATCC (Manassas, VA, USA), while a custom gas mixture of 20.9% oxygen/79.1% nitrogen was acquired from Indiana Oxygen (Indianapolis, IN, USA). Povidone-iodine swabs were purchased from Fisher Scientific (Hampton, NH, USA). Ophthalmic gel was obtained from Amazon (Systane Lub Eye Nighttime Ointment, Amazon, Seattle, WA, USA). Isoflurane was purchased from Patterson Veterinary (Loveland, CO, USA), while buprenorphine SR was purchased from ZooPharm (Fort Collins, CO, USA). PdBMAP was generously provided by Profusa, Inc. (Emeryville, CA).

### Methods

### Preparation of Regenerated Silk Fibroin Solution

Bombyx mori silk cocoons were degummed according to previously described methods.[34] To remove sericin, the cocoons were placed into 0.02 M NaCO₃ and boiled for 30 minutes. The silk solution was left to dry overnight before the silk fibroin was dissolved in 9.3 M LiBr. This solution was heated to 60° C. for 4 hr, and then regenerated cellulose tubing with a molecular weight cut-off of 3.5 kDa (Spectrum Laboratories Inc., Rancho Dominguez, CA, USA) was used to dialyze the silk solution. Post-dialysis, the silk was frozen overnight at −80° C. and then lyophilized for two days. The purified silk fibroin was then dissolved in 1,1,1,3,3,3-hexafluoro-2-propanol (HFIP) at 17 w/v % for 24 hours with periodic agitation throughout.

### Fabrication of HFIP-Based Silk Films

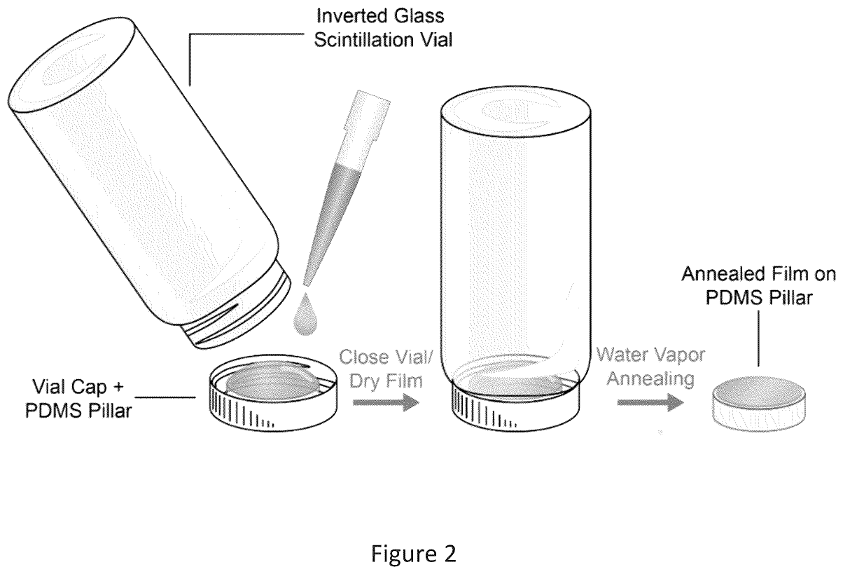

To enable film fabrication, the concentrated silk fibroin solution was diluted to 5 w/v % and 10 w/v % silk in HFIP. A 10 mg/mL PdBMAP in HFIP solution was added to these silk solutions in the appropriate quantity to obtain a concentration of 0.1 w/w % PdBMAP relative to the mass of the silk, and the resulting solution was vortexed for 30 seconds to ensure homogenous distribution. The film drop-casting setup consisted of a glass scintillation vial inverted with a 12 cm PDMS pillar placed on the interior of the cap (FIG. 2). This environment was designed to slow evaporation of the volatile solvent to ensure homogenous film topography and avoid film warping. Then 100 μL of the silk solution was pipetted onto the PDMS pillars and the glass vial was loosely screwed on top to facilitate slow evaporation. The vials were left overnight in a fume hood to dry. The dried films were then rendered water-insoluble via water vapor annealing, a process in which the presence of water vapor plasticizes the silk fibroin and induces crystalline β-sheet formation. This process has been described in more detail elsewhere. Briefly, the films were added to a vacuum chamber with a bath of deionized water, and the chamber was degassed to −25 mmHg gauge pressure. The films were left overnight before slowly releasing the vacuum and then allowing them to dry.

### Fabrication of HFIP-Based Silk Sponges

The silk sponges were fabricated from an adaptation of a previously established protocol.[40] In brief, NaCl was sieved to attain crystals between 100 and 450 μm. Then 3.4 g of the sieved crystals were added to 30 mm diameter glass vials to form a level bed at the base of the vial. Then 10 mg/mL PdBMAP in HFIP was added to a 17 w/v % silk in HFIP solution to achieve 0.1 w/w % PdBMAP compared to the silk content, and the solution was vortexed for 30 seconds. Next 1 mL of this silk solution was cast over the salt crystals and the vessel tightly sealed to prevent evaporation of the volatile solvent. To ensure homogenous distribution of the silk, the solution was left overnight to penetrate the entire salt bed. Afterward, the cap of the vial was removed, allowing the solvent to evaporate overnight to leave behind a solid silk network surrounding the salt crystals. The solid product was water-vapor annealed in the same manner as the films to render the silk network water-insoluble due to beta sheet formation via self-assembly. The annealed silk network was dialyzed in deionized water for 3 days with twice daily water changes to dissolve the salt leaving a porous silk sponge. For the in vitro photophysical characterization and in vivo implantation, sponges were sliced into 1 mm thick discs using a razor blade and a blade guide prior to analysis.

### Scanning Electron Microscopy

Scanning electron microscopy (SEM) imaging was performed on the silk films and sponges with and without PdBMAP using an EVO MA10 (Zeiss, Oberkochen, Germany) in order to examine surface morphology, porosity, and cross-section thickness (in the case of films). Films and sponges were first placed in liquid nitrogen for 30 seconds, then samples were cut to the desired size with a cooled razor blade. Subsequently, samples were adhered to SEM stubs with carbon adhesive. Prior to imaging, samples were sputter coated in gold for 2 minutes. Samples were then imaged using an accelerating voltage of 10 kV.

### In Vitro Enzymatic Degradation Study

PdBMAP-containing 5 w/v % silk films, 10 w/v % silk films, and sponges were placed into 24-well plates in 2 mL of 1 U/mL protease XIV in PBS or 2 mL of 1 U/mL α-chymotrypsin in PBS (n=3 for each condition). Samples were aged at 37° C. with the enzyme solution being replaced every other day. At select time points (days 0, 1, 4, 7, 14), samples were removed, thoroughly washed with distilled water 3 times for 30 min., frozen at −80° C., and lyophilized for 48 hr. The mass of the lyophilized samples was then compared to the initial mass of the dry samples.

### Metabolic Activity and Live/Dead Assay

A 1 v/v % antibiotic-antimycotic and 10 v/v % FBS were added to high-glucose DMEM which was used to culture human dermal fibroblasts. Every three days, the media was replaced. Films and sponges were sterilized by autoclaving the dried samples. Seeding of the fibroblasts took place at a concentration of 5,000 cells/cm². At select time points (1, 3, and 7 days), cells were added to culture media with 10 v/v % AlamarBlue for 3 hours at 37° C., then 150 μL of the supernatant was removed and placed into opaque 96-well plates. Metabolic activity was assessed with a SpectraMax M2 multi-node microplate reader (Molecular Devices, San Jose, CA, USA) (λ_{Ex}: 560 nm, λ_{Ex}: 590 nm), and the fluorescence output was compared to the day 1 results (n=5).

After 7 days, a live/dead assay was performed by staining cells with ethidium homodimer-1 (EthD-1) and calcein acetoxymethyl ester (calcein AM) for 15 minutes and then rinsing with PBS. Samples were imaged with a BZ-X700 fluorescence microscope (Keyence Corp., Itasca, IL, USA). Cell viability was then assessed using CellProfiler software (Broad Institute, Boston, MA, USA) (n=5).

### In Vitro Assessment of Oxygen Sensing Capabilities

A custom fluorimeter setup that is described in more detail elsewhere[16,41] was used to assess the oxygen sensing properties of PdBMAP-containing samples in 37° C. PBS. Photophysical assessment was performed using an Edinburgh Instruments FLS1000 Photoluminescence Spectrometer (Livingston, Scotland) equipped with a double Czerny-Turner excitation monochromator, a single Czerny-Turner emission monochromator, a photomultiplier tube (PMT) detector, a 450 W xenon arc lamp, and a pulsed xenon microsecond flashlamp. For all measurements, a 715 nm long-pass filter was placed in front of the emission monochromator, and the PMT detector was cooled to −22° C. Either notched or notchless custom cuvette inserts[41] were used to hold the silk-PdBMAP sponge or film, respectively, across the diagonal of a quartz cuvette containing PBS. The quartz cuvette was added to a water-jacketed sample holder, and a recirculating bath was used to control the temperature to 37° C. The sample was oriented in a back-scattered geometry relative to the excitation source and detector which were perpendicular to one another. First, steady-measurements of the emission spectra (taken with λ_{Ex} of 630 nm) and excitation spectra (taken at λ_{Em} of 804-805 nm) in aerated PBS were obtained. Then, tubing was inserted into the custom cuvette inserts to modulate the dissolved oxygen concentration. MC series Alicat mass flow controllers (Tucson, AZ, USA) were used to combine N2 and a custom 20.9% O₂/79.1% N₂ mixture. The desired oxygen concentrations (0.5, 1, 2, 5, or 10% O₂) were bubbled into the PBS, while a Unisense needle-based micro Clark electrode (Aarhus, Denmark) continuously monitored the solution. For each oxygen concentration, bubbling was done for at least 10 minutes before any lifetime measurements were taken. During this time, continuous Clark electrode readings were monitored alongside occasional measurements of the signal output intensity (Ex: 630 nm, λ_{Em}: 804-805 nm) to ensure that conditions had plateaued. The phosphorescence lifetime decay at a given dissolved oxygen concentration was then obtained by exciting the sample with 630 nm using the microsecond flashlamp operating at 40 Hz. The emission was then monitored at the maximum emission wavelength (λ_{Em}: 804-805 nm). Decays were obtained using 4,000 data channels and 5,000 counts at the maximum intensity. Lastly, instrument response function (IRF) decay curves (λ_{Ex}, λ_{Em}: 630 nm) were obtained after the removal of the 715 nm long-pass filter and the reduction of excitation intensity to 1%.

Using the obtained IRF curves, the phosphorescence decays of the samples at various oxygen concentrations were fit according to a reconvolution fit in Origin (OriginLab Corporation, Northampton, MA, USA). Fitting was performed using all data until the point at which the maximum intensity dropped to ˜1/e⁴ of its initial value. Depending on the nature of each decay curve, a customized version of the fitconv algorithm[42] was used to fit the sample decay to either a monoexponential (Eq. 4) and/or biexponential (Eq. 5) equation. Here, I is the signal intensity, while I_{bkgd} is the background intensity. A is the pre-exponential factor of a given lifetime (T), and t denotes time.

$$I = A exp ( - t / T ) + {I}_{b k g d} \qquad ( 4 )$$

$$I = {A}_{1} exp ( - t / {T}_{1} ) + {A}_{2} exp ( - t / {T}_{2} ) + {I}_{b k g d} \qquad ( 5 )$$

For biexponential fits, the amplitude average lifetime, Tₘ, and fractional contributions, f₁ and f₂, were assessed (Eqs. 6 and 7, respectively). Note that there are multiple variants of average lifetimes, but this is the correct version to use in this instance.[17,43]

$${T}_{m} = ( {A}_{1} {T}_{1} + {A}_{2} {T}_{2} ) / ( {A}_{1} + {A}_{2} ) \qquad ( 6 )$$

$${f}_{1} = {A}_{1} {T}_{1} / ( {A}_{1} {T}_{1} + {A}_{2} {T}_{2} ) , {f}_{2} = 1 - {f}_{1} \qquad ( 7 )$$

Dissolved oxygen concentrations were determined by taking into account the solution temperature and salinity, as well as the concentration of the bubbled gas. The deaerated lifetime was obtained from a predictive method as described elsewhere[16]. Depending on whether the Stern-Volmer plot (T₀/Tₘ or T₀/T versus dissolved oxygen concentration) appeared to be linear or downward-curving, either the linear Stern-Volmer equation (Eq. 1) or the two-site model (Eq. 3) was applied, respectively.

The same fluorimeter setup was used to aid photophysical assessment during an in vitro degradation procedure. In this case, 10 w/v % silk film and sponge (˜1 mm thick) samples containing PdBMAP were assessed initially and after 4 days of exposure to 15 mL of 37° C. DBPS+1 U/mL α-chymotrypsin (n=2 for each condition). The enzyme solution was exchanged midway through the experiment at the two-day mark. After the exposure, samples were thoroughly rinsed in distilled water. Before and after enzymatic exposure, samples were assessed in 37° C. PBS according to the described procedure above with the exception that decays were only collected at 3 and 20.9% O₂ (aerated PBS).

### In Vivo Implantation of Silk Films and Sponges

All animal studies were conducted according to protocols that were reviewed and approved by both the Tufts Institutional Animal Care and Use Committee (M2019-121) and the Air Force Surgeon General's Office of Research Oversight and Compliance. These animal studies were performed in a manner that was consistent with the principles described by both the Animal Welfare Act and the “Guide for the Care and Use of Laboratory Animals” (Institute of Laboratory Animal Research, National Research Council, National Academies Press, 2011).

The 5 and 10 w/v % silk films with and without PdBMAP were fabricated as described earlier but using smaller diameter PDMS pillars (5 versus 12 mm). Silk sponges with and without PdBMAP were fabricated using the protocol described above and then were cut into small 5 mm discs using a biopsy punch. A two hour ethylene oxide exposure at room temperature was used to sterilize the samples. Following this exposure, the ethylene oxide chamber was vented overnight to degas the residual ethylene oxide. Prior to implantation, initial anesthetization of Sprague-Dawley rats (Charles River Laboratories, Cambridge, MA, USA) was achieved with 1 L/min of total gas flow through a nose cone. Initially, the flow was 1.5-3 v/v % isoflurane, and the remainder of the feed was house oxygen. Once the animal remained asleep and displayed no pain response to pressure applied to the foot, the isoflurane concentration was lowered to approximately 1-2 v/v %. The implantation sites were then shaved, and ophthalmic gel was added to the eyes to prevent corneal drying. Then, buprenorphine SR was administered at a concentration of 1 mg/kg of body weight. While the rats were on a heating pad at 37° C., the implantation site was cleaned with both povidone-iodine and alcohol wipes. A pocket into the subcutaneous layer was formed by first making four incisions with a length of ˜8 mm each. Forceps were used to raise the skin above this pocket so that the sample could be placed into the subcutaneous pocket. The four samples implanted were placed >3 cm apart to prevent crosstalk between the PdBMAP-containing film and sponge. Furthermore, PdBMAP-containing samples were placed on opposite ends of the animal dorsal for this reason. The incisions were stitched shut with a wound clip. The wound was cleaned, and the animal was removed from the anesthesia and monitored while waking up. The wound clips were removed 7 days post-implantation. Post-operation, animals were monitored once daily for three days and then once weekly for the proceeding weeks.

### Histology of Implanted Silk Films and Sponges

At select time points (7, 14, and 28 days), a CO₂ exposure was used to euthanize a rat for histology in compliance with 2020 American Veterinary Medical Association (AVMA) guidelines (n=1 per time point). During the euthanasia process, each rat was monitored for cardiac arrest at which point the exposure was continued for an additional minute. Then, cervical dislocation was applied as an additional euthanasia method. Histology sections were obtained from explants (˜4 cm² diameter skin sections at the implantation sites). The removed skin sections were washed with PBS, placed in 4% buffered paraformaldehyde for an hour, washed with PBS again, placed in a solution of 15% sucrose in PBS for four hours, and then placed in a solution of 30% sucrose overnight. Samples were then removed and coated in OCT compound before being placed in a bath of liquid nitrogen-cooled isopentane. Once frozen, samples were removed and stored at −80° C. A cryostat was used to obtain tissue cross-sections with a thickness of ˜12 mm. These sections were attached to Tissue Path Superfrost Plus gold slides followed by an additional tissue fixation step via a 15 minute exposure to 4% buffered paraformaldehyde. Fixed samples were rinsed with distilled water, then stained with H&E. Imaging of tissue sections was performed with a BZ-X700 fluorescence microscope (Keyence, Itasca, IL, USA) using brightfield settings.

### In Vivo Assessment of Oxygen-Sensing Capabilities of Implanted Silk Films and Sponges

The oxygen-sensing capabilities of implanted PdBMAP-containing 10 w/v % silk films and silk sponges was assessed at select time points (14, 18, 21, 25, and 28 days). These experiments were not started until day 14 to provide sufficient time for wound clips to be removed and for incision wounds to heal. A Beacon system and associated readers (Profusa, Inc., Emeryville, CA, USA) were used to obtain lifetime decays of the implanted PdBMAP-containing silk samples. Lifetime decays were obtained every five seconds, while each decay was an average of four acquisitions performed in succession of one another. On each day that sensor performance was evaluated, the lifetime of the sensor was first monitored while the rat was under anesthesia. The anesthesia administered via the nose cone provided a hyperoxic condition as it was 1-2 v/v % isoflurane in house oxygen. The lifetime was monitored versus time under this condition and then the nose cone was briefly removed for 1 min and subsequently reattached without the animal waking up. During the time period when the nose cone was removed, the rat was breathing in atmospheric oxygen providing a normoxic condition. On the last day, these two rats were euthanized via a CO₂ exposure in compliance with the 2020 AVMA recommendations. However, during this process, one sample on each rat was monitored using the Beacon system. First, the nose cone was taken off the rat, and the animal was placed in an open chamber that was initially under atmospheric conditions. Collection of lifetime decays with the Beacon system and reader started at this time and continued throughout the euthanasia process. After one minute, the chamber was closed, and CO₂ was administered into the cage via a feed line. The animal experienced hypoxic conditions due to this CO₂ exposure. This exposure continued for one minute after cardiac arrest was noted, then cervical dislocation was applied as an additional euthanasia method. In all cases, a program developed in Python was used to fit the obtained lifetime decay curves. In this manner, a monoexponential (Eq. 4) tail-fit was applied to the region of data between 15 μs after the maximum intensity and the point at which the 3.5 e-folds of decay occurred.

### Results

### Sample Fabrication

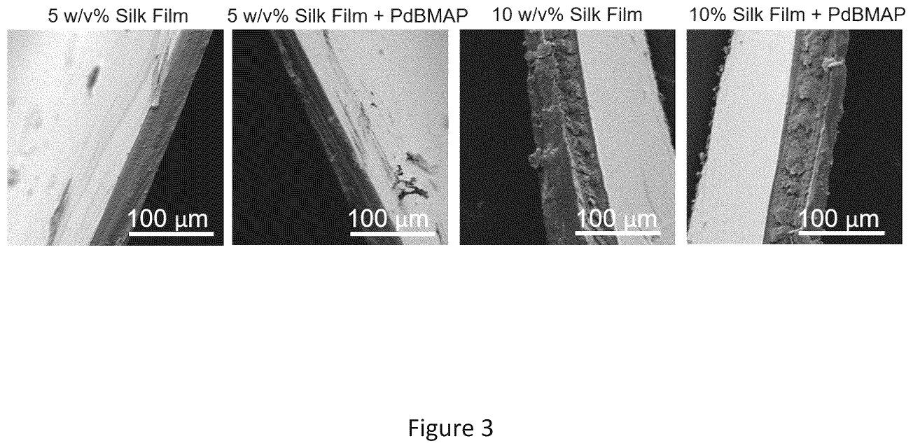

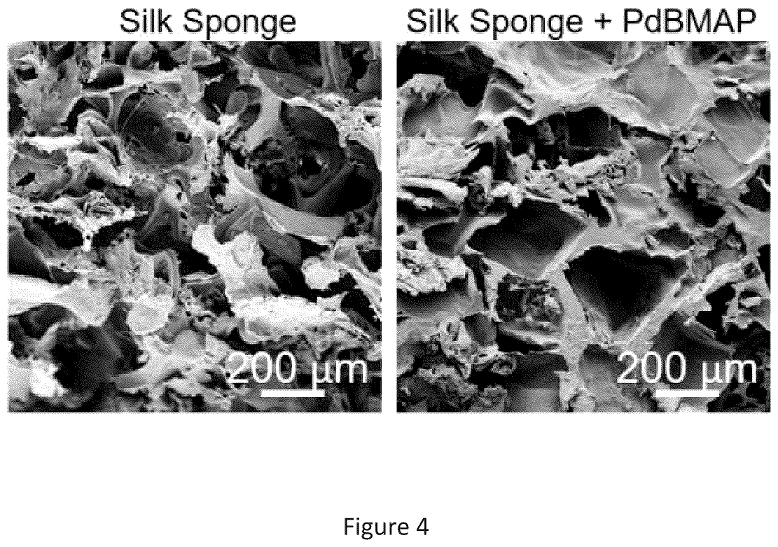

PdBMAP-containing silk films and sponges were successfully fabricated using HFIP as the processing solvent (FIG. 1). In both cases, water vapor annealing was used to induce β-sheet formation, a necessary step to render the silk matrix water-insoluble via physical crosslinks.[39] Films were fabricated by drop casting the silk/chromophore solution onto a mold which was then dried to produce a rigid and flat film that was homogenously transparent and green (FIG. 1B). By maintaining the volume of the cast solution but altering silk concentration, the film thickness was varied (FIG. 3, ˜40 or 80 μm for 5 or 10 w/v % silk solution, respectively). Three-dimensional, porous silk sponges were produced by first casting the initial silk solution over NaCl particles that were subsequently leached out of the scaffolds after water vapor annealing, creating an interconnected porous network (FIG. 4). A schematic of the fabrication process for both sponges and films is shown in FIG. 1C.

### Cytocompatibility

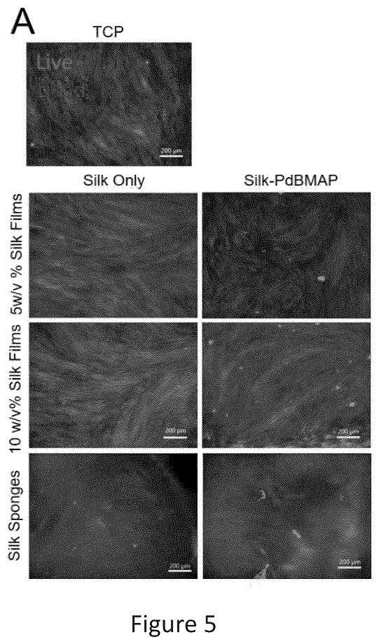

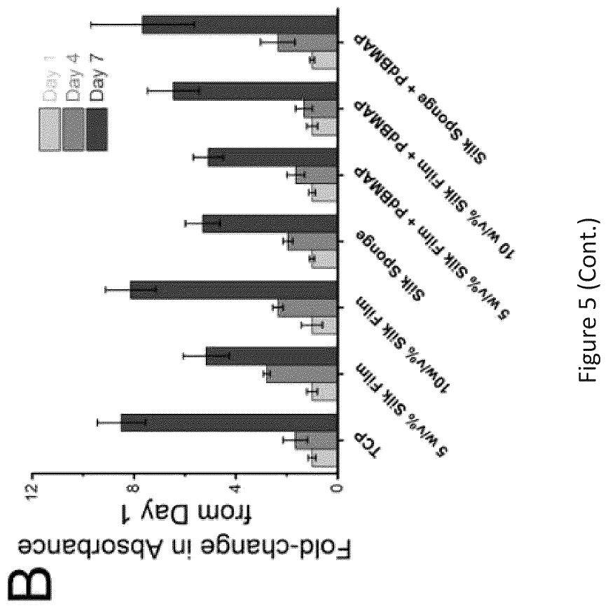

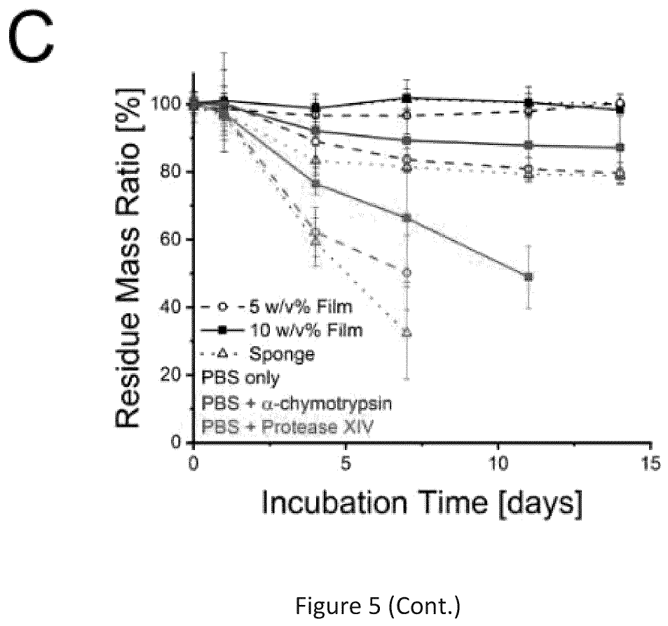

Both silk-only films and sponges and PdBMAP-containing equivalents were seeded with human dermal fibroblast cells and observed to be cytocompatible. Live/Dead imaging after 7 days showed that dermal fibroblast cells readily spread onto the silk substrates, and >86% of cells were viable (FIG. 5A). The accompanying assessment of metabolic activity demonstrated increased cell activity on the materials over time (FIG. 5B).

### In Vitro Degradation

Enzymatic-based in vitro degradation studies demonstrated that mass loss of the materials was related to the enzyme used and the form of the silk matrix as expected (FIG. 5C). For a specific silk composition, degradation occurred but was slower in the presence of α-chymotrypsin than protease XIV. Comparing the silk films, the thinner ˜40 μm film prepared from the 5 w/v % silk precursor solution exhibited more rapid mass loss compared to the ˜80 μm thick 10 w/v % silk counterpart. For both enzyme solutions, the porous sponges exhibited a greater mass loss than either of the films due to the higher surface area for enzymatic access to initiate the degradation process.

### In Vitro Dissolved Oxygen-Sensing Properties

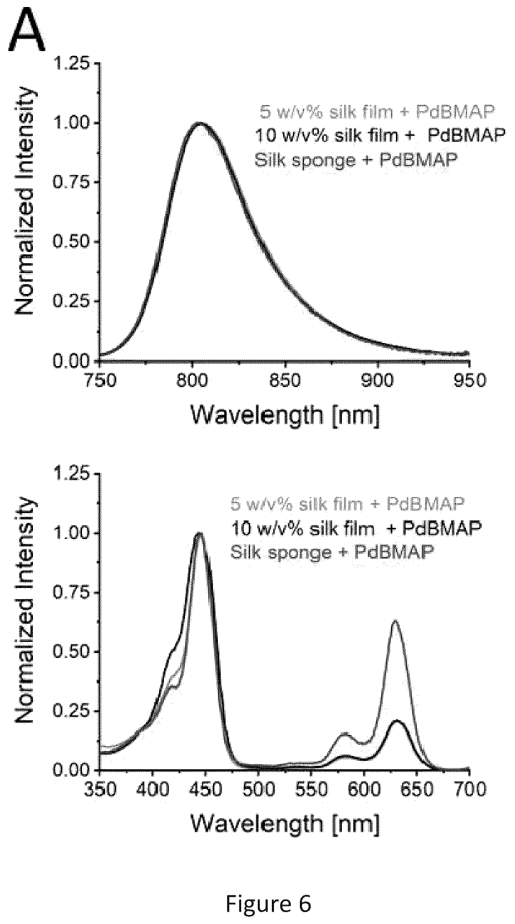

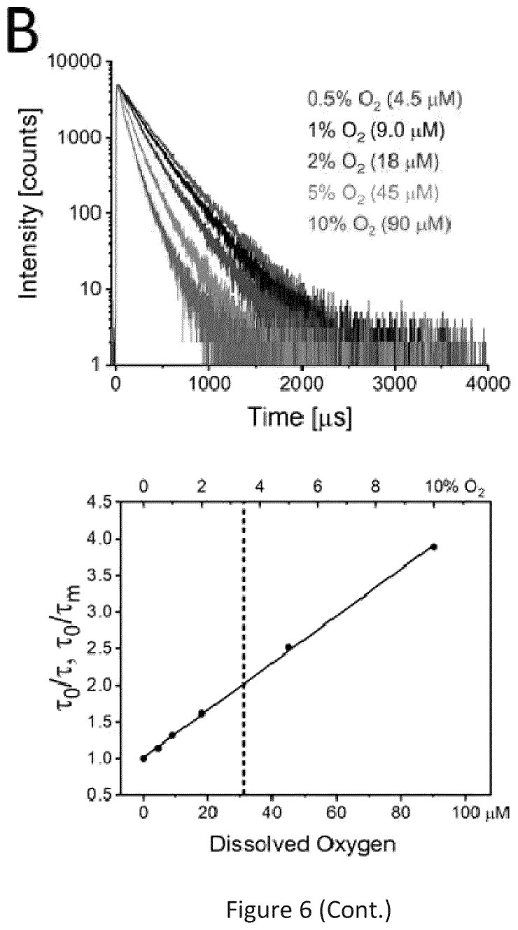

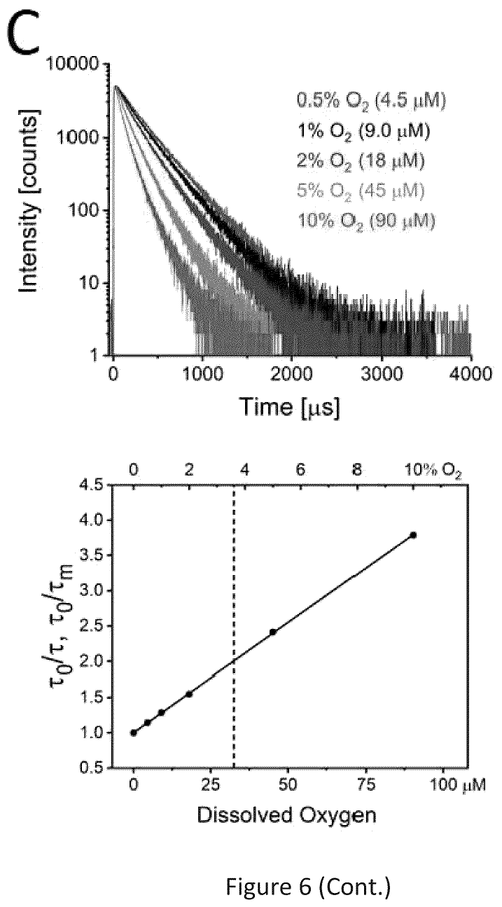

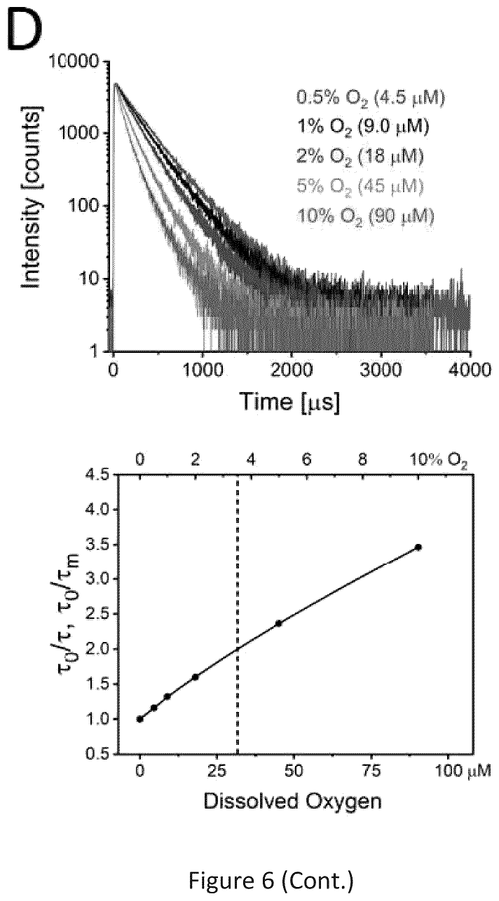

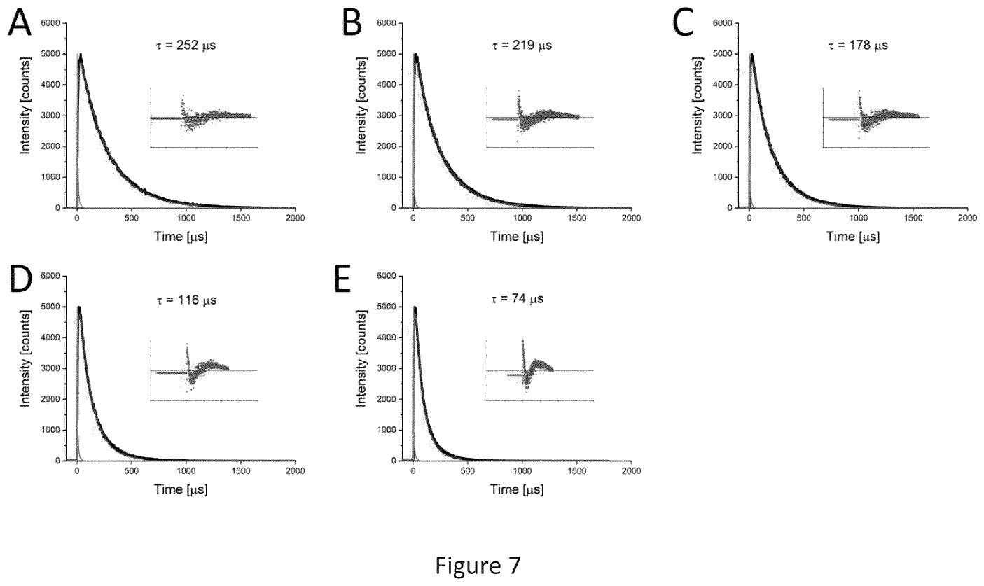

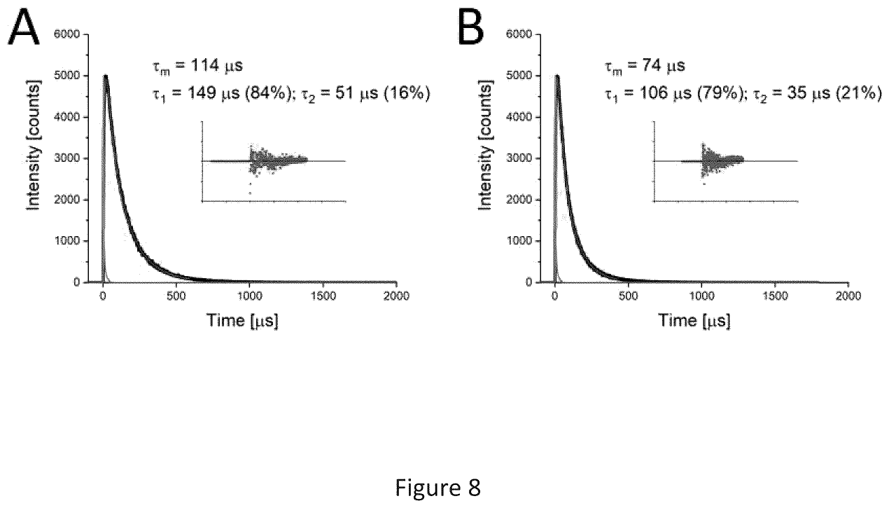

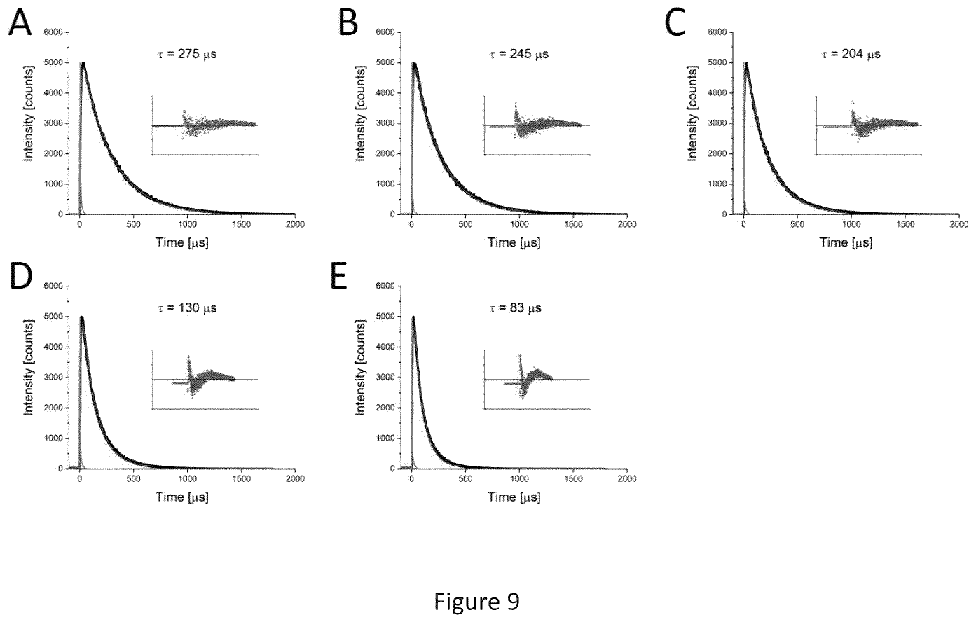

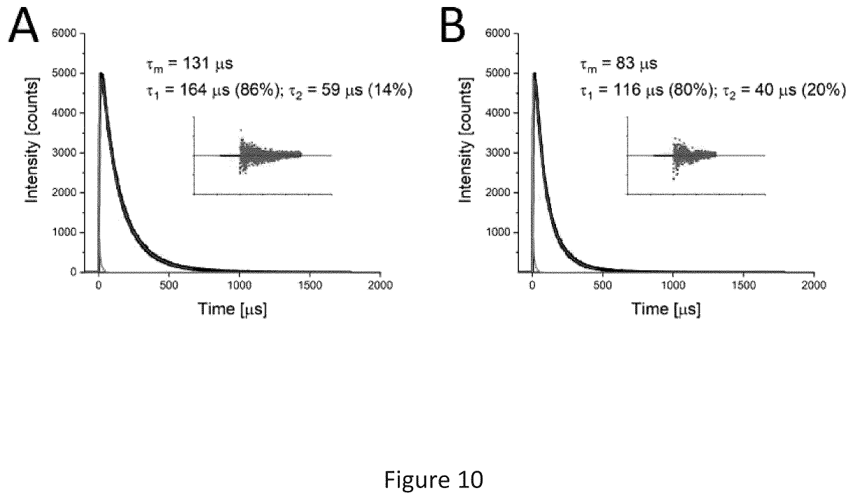

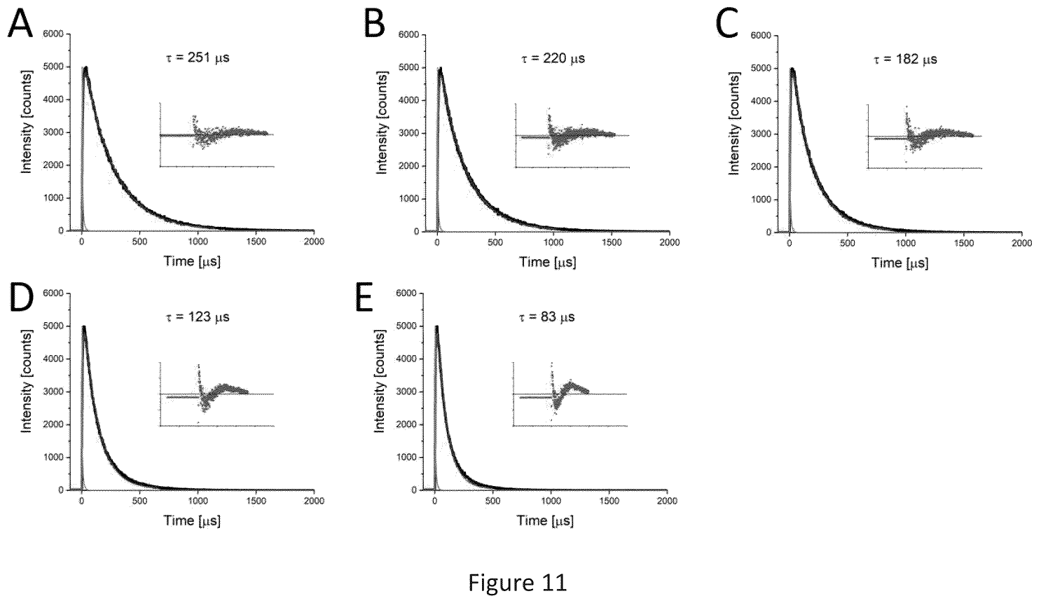

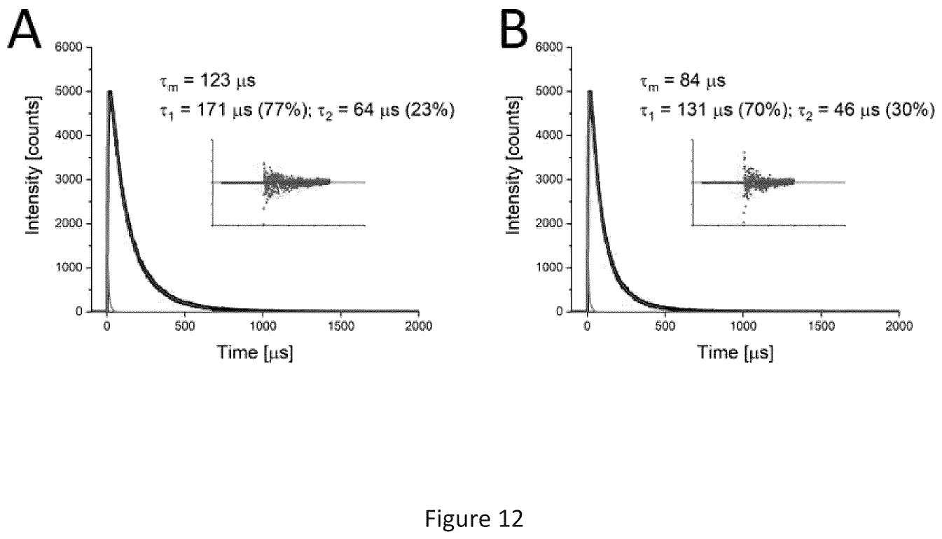

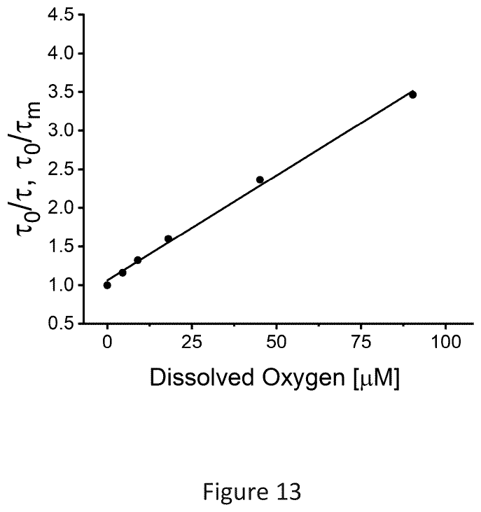

The oxygen-sensing properties of the silk films and sponges were investigated in 37° C. PBS (FIGS. 6-12, Tables 1-4). Absorption (444-446 nm, 629-631 nm) and emission (804-805 nm) maxima were similar to those observed for the chromophore in solution.[16] The phosphorescence lifetime of the near-infrared emission was quenched as the concentration of dissolved oxygen in the surrounding solution increased. For all samples, a biexponential reconvolution fit either did not converge or offered minimal fit improvement <˜45 μM dissolved oxygen (5% (2), so a monoexponential fit was deemed sufficient at these concentrations (FIGS. 7, 9, and 11). A slight deviation from monoexponential character became more evident at higher dissolved oxygen concentrations, so the average lifetime derived from a biexponential fit was used for 5 and 10% O₂ measurements (FIGS. 8, 10, and 12). However, a monoexponential fit was within 0.1-1.2% of the average lifetime in all cases (Tables 2-4). Stern-Volmer plots were approximately linear for the silk film samples and slightly downward-curving for the silk sponge sample. Regardless, a linear Stern-Volmer plot for the sponge can reasonably approximate its behavior (R²=0.997, FIG. 13). The overall oxygen-sensing properties of all interrogated samples were remarkably similar. The deaerated lifetime was ˜300 μs, while the Stern-Volmer quenching rate constant ranged from 2.7-3.2×10⁴ M⁻¹. In all cases, the lifetime was quenched to 50% of its maximum value at ˜3.4-3.5% O₂ (˜31-32 μM).

TABLE 1
Select oxygen-sensing properties of PdBMAP-containing silk films and sponges in 37°
C. PBS. Values in parentheses after K_{SV, 1} and K_{SV, 2} denote the fraction of each component.

| | | | | | | | | DO |
|---|---|---|---|---|---|---|---|---|
| | | | | | | | | [μM] |
| | | | | | | | | for |
| | λ_{Abs} | λ_{Em} | τ₀ | k_{q} | K_{SV} | K_{SV, 1} | K_{SV, 2} | which |
| | [nm] | [nm] | [μs] | [M⁻¹s⁻¹] | [M⁻¹] | [M⁻¹] | [M⁻¹] | τ = 0.5τ₀ |
| 5 w/v % silk | 446,631 | 805 | 288 | 1.1E+08 | 3.2E+04 | — | — | 31 |
| film | | | | | | | | |
| 10 w/v % | 444,631 | 805 | 315 | 9.8E+07 | 3.1E+04 | — | — | 32 |
| silk film | | | | | | | | |
| Silk sponge | 446,629 | 804 | 291 | 9.3E+07* | 2.7E+04* | 4.6 × | 6.9 × | 32 |
| | | | | | | 10⁴ | 10³ | |
| | | | | | | (0.78) | (0.22) | |

*Estimated from a linear fit of the Stern-Volmer plot.

TABLE 2
Calculated lifetimes for 5 w/v % silk +
PdBMAP film using monoexponential (τ) and/
or biexponential (τₘ) fits. DO = dissolved oxygen.

| DO | DO | τ | τₘ | τ₁ | τ₂ |
|---|---|---|---|---|---|
| [μM] | [%] | [μs] | [μs] | [μs] | [2 μs] |
| 4.51 | 0.5 | 252 | — | — | — |
| 9.0 | 1.0 | 219 | — | — | — |
| 18.1 | 2.0 | 178 | — | — | — |
| 45.1 | 5.0 | 116 | 114 | 149 (84%) | 51 (16%) |
| 90.3 | 10 | 74 | 74 | 106 (79%) | 35 (21%) |

TABLE 3
Calculated lifetimes for 10 w/v % silk +
PdBMAP film using monoexponential
(τ) and/or biexponential (τₘ) fits. DO = dissolved oxygen.

| DO | DO | τ | τₘ | τ₁ | τ₂ |
|---|---|---|---|---|---|
| [μM] | [%] | [μs] | [μs] | [μs] | [2 μs] |
| 4.51 | 0.5 | 275 | — | — | — |
| 9.0 | 1.0 | 245 | — | — | — |
| 18.1 | 2.0 | 204 | — | — | — |
| 45.1 | 5.0 | 130 | 131 | 164 (86%) | 59 (14%) |
| 90.3 | 10 | 83 | 83 | 116 (80%) | 40 (20%) |

TABLE 4
Calculated lifetimes for silk-PdBMAP
sponge using monoexponential (τ) and
biexponential (τₘ) fits. DO = dissolved oxygen.

| DO | DO | τ | τₘ | τ₁ | τ₂ |
|---|---|---|---|---|---|
| [μM] | [%] | [μs] | [μs] | [μs] | [2 μs] |
| 4.51 | 0.5 | 251 | — | — | — |
| 9.0 | 1.0 | 220 | — | — | — |
| 18.1 | 2.0 | 182 | — | — | — |
| 45.1 | 5.0 | 123 | 123 | 171 (77%) | 64 (23%) |
| 90.3 | 10 | 83 | 84 | 131 (70%) | 46 (30%) |

Note: For all compositions, the deaerated lifetime was estimated through a linear predictive method. This methodology was chosen as a result of the difficulty in attaining 0 μM dissolved oxygen during the lifetime measurement and the ability of even very small quantities of dissolved oxygen to quench the response. These difficulties were exacerbated by the small volume of solution and the necessity to continuously bubble without disrupting either the sponge or film sample or the measurement. This method involves plotting the inverse of the lifetime as a function of dissolved oxygen concentration. Then, a least squares linear regression was used to acquire the intercept which should equal the inverse of the deaerated lifetime in accordance with the Stern-Volmer equation. This method was applied using 0.5-5% O₂ for the silk film samples and 0.5-1% O₂ for the sponge samples. A smaller region was used for the sponge samples considering the downward-curving Stern-Volmer plot. This method should still be applicable as even the initial region of downward-curving Stern-Volmer plots is typically linear.

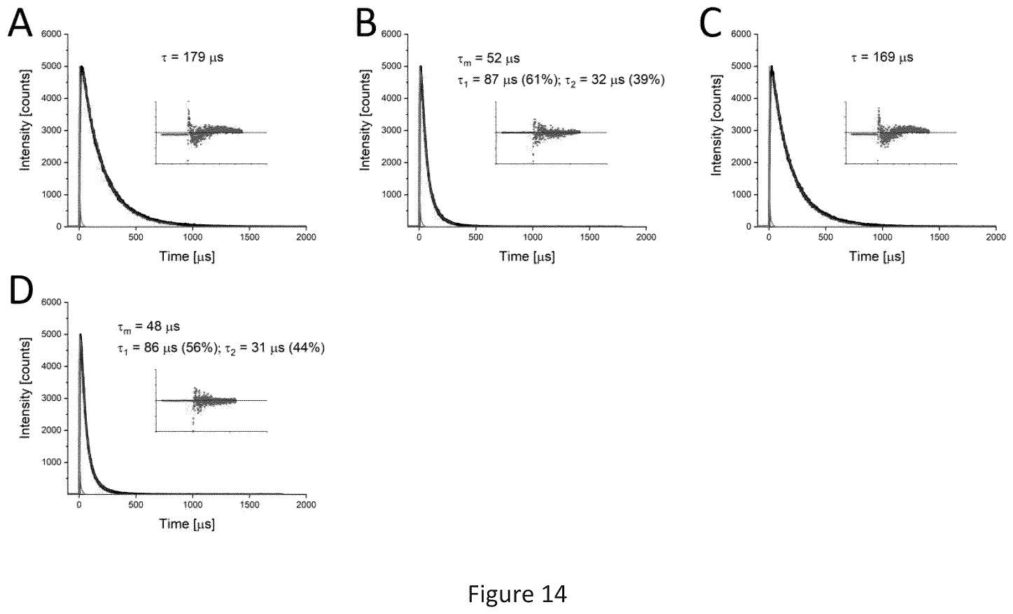

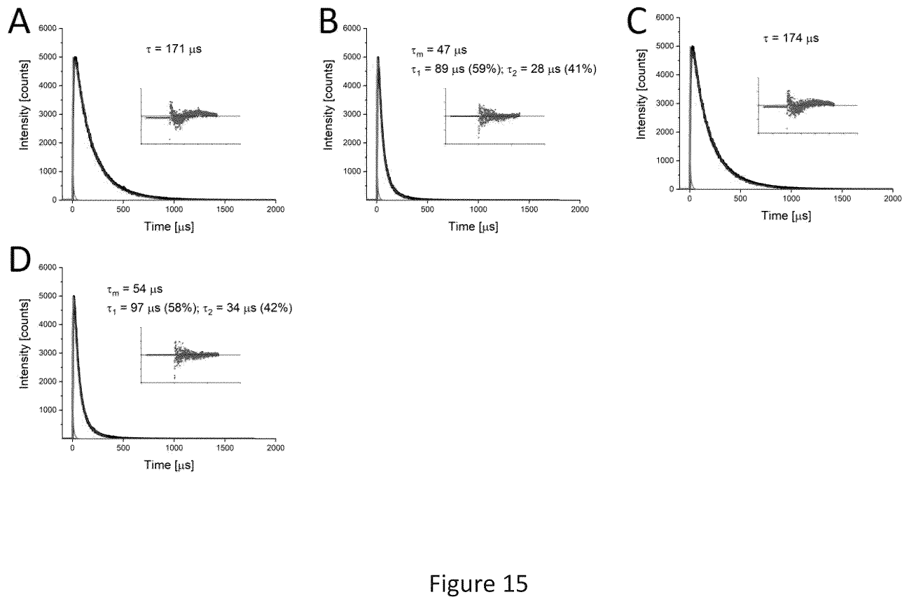

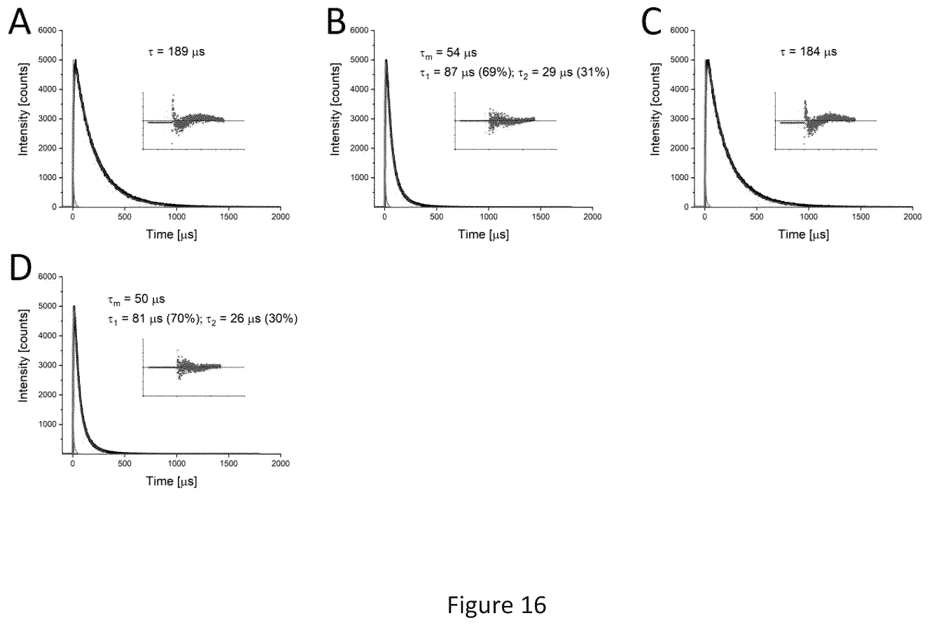

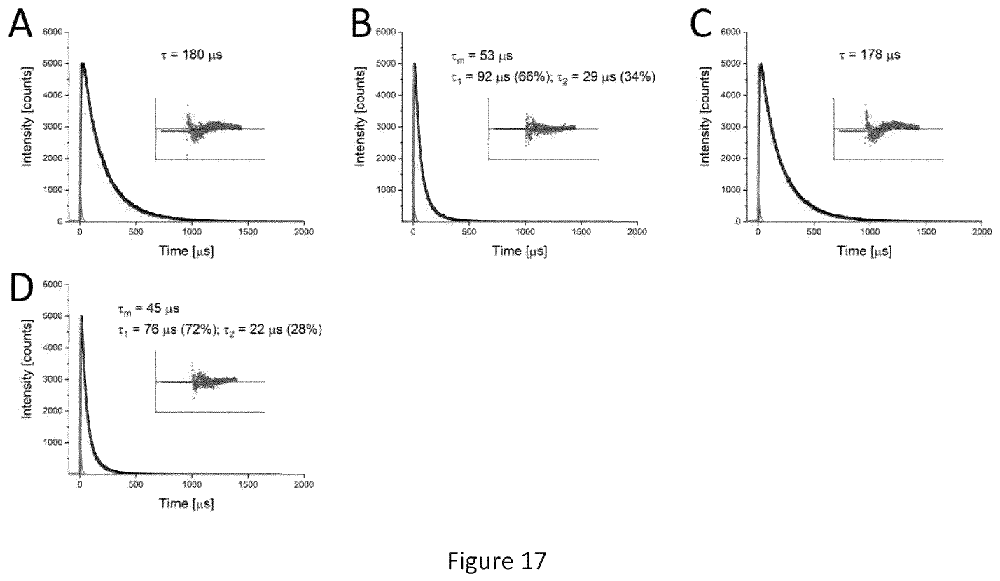

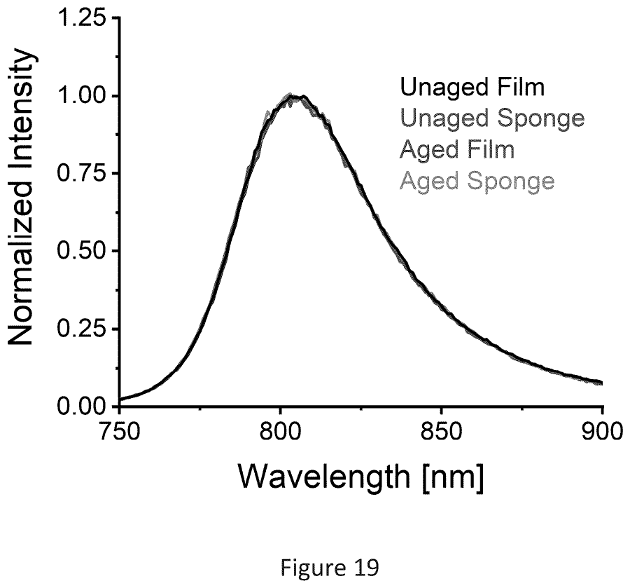

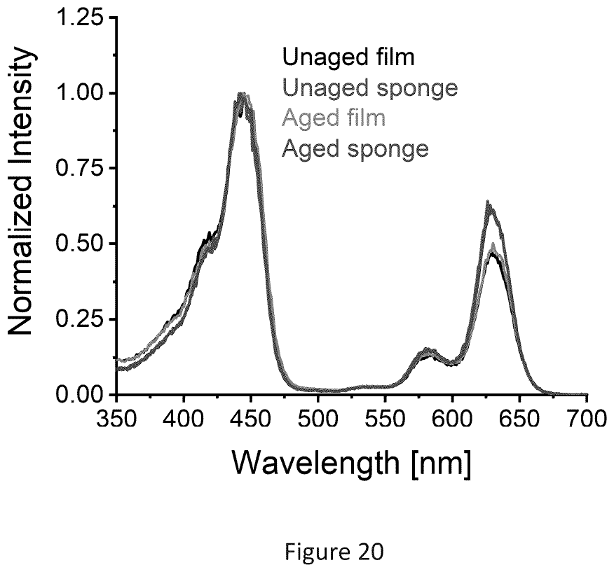

Further studies focused on the behavior of the 10% w/v silk film (down-selected for its longer degradation time) versus the sponge. Additional in vitro experiments demonstrated that these compositions retained oxygen-sensing capabilities after a 4-day exposure to 37° C. PBS+1 U/mL α-chymotrypsin. Lifetimes at 3 and 20.9% O₂ were not substantially altered by the enzyme exposure (FIGS. 14-18), and emission and excitation spectra were virtually unchanged (FIGS. 19-20).

### In Vivo Performance

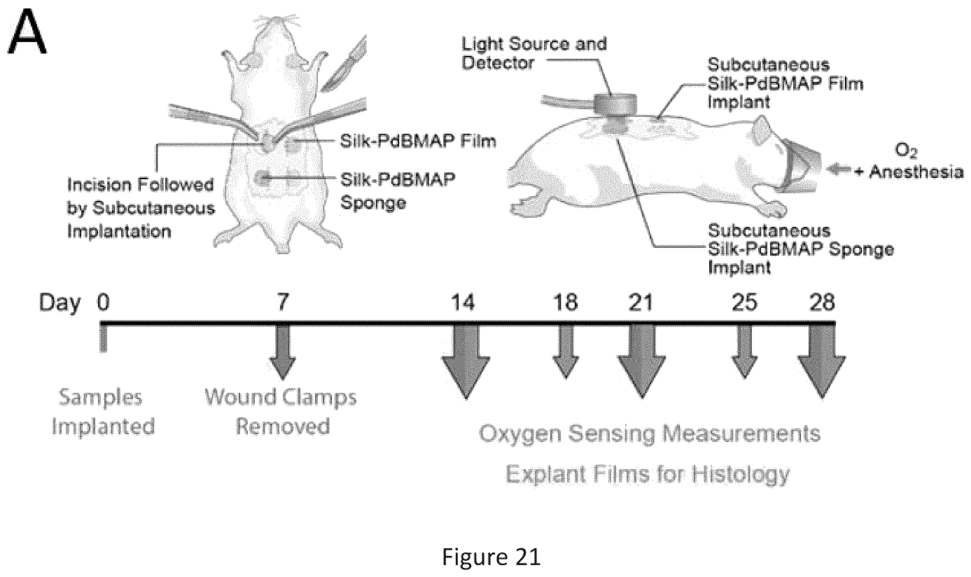

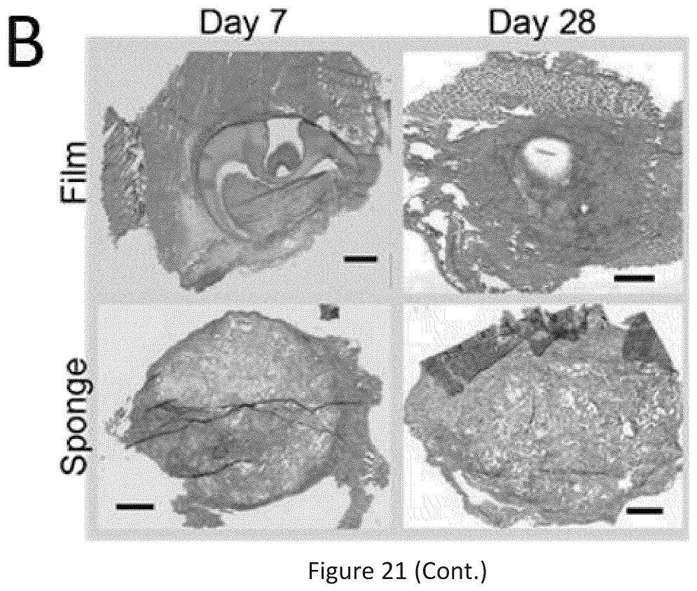

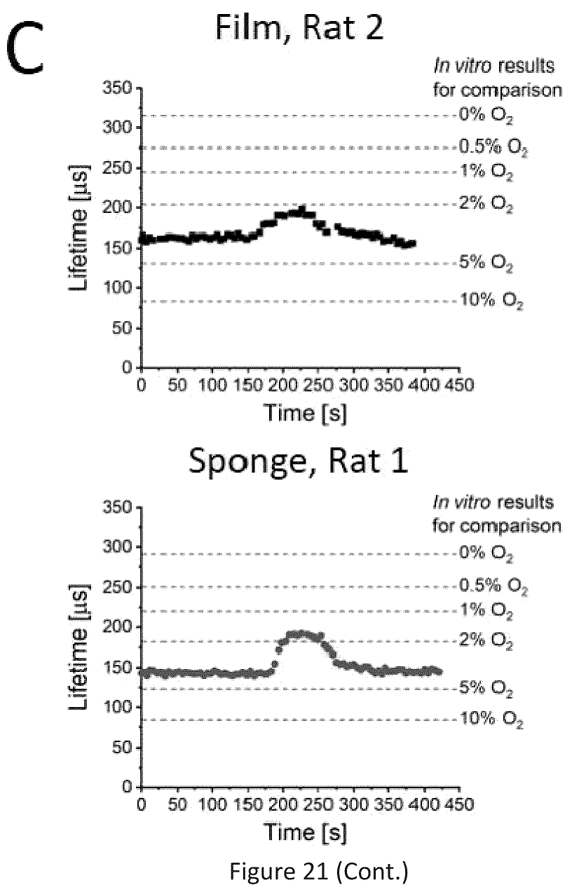

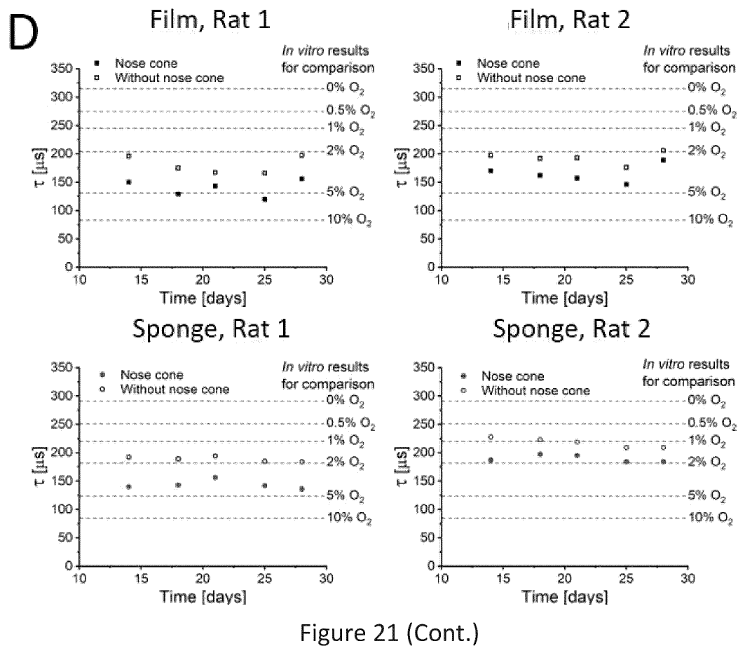

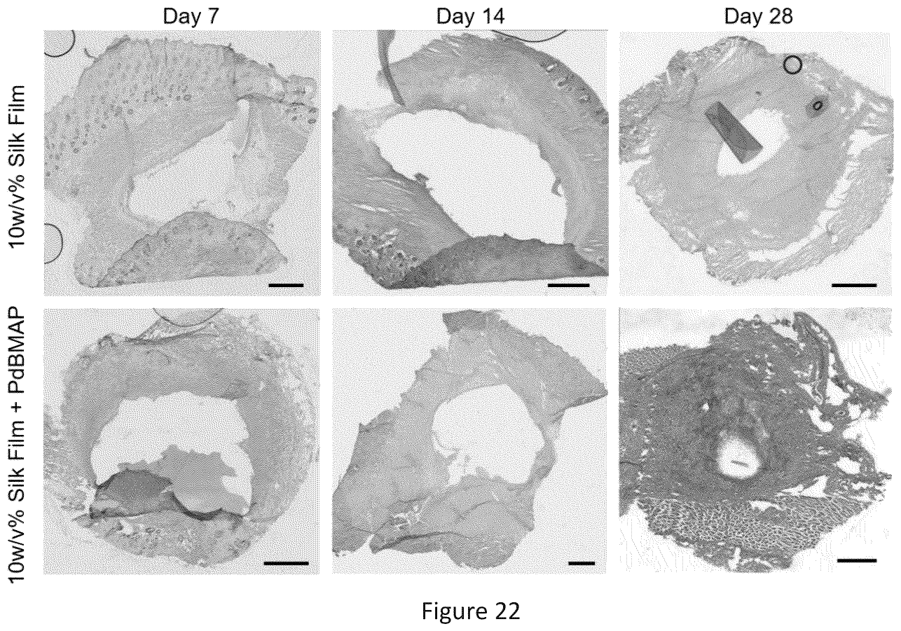

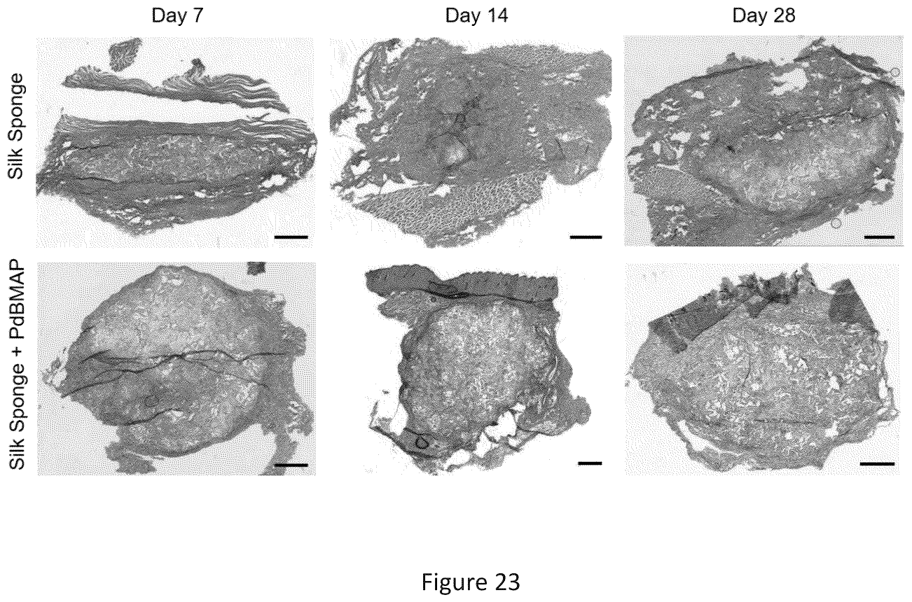

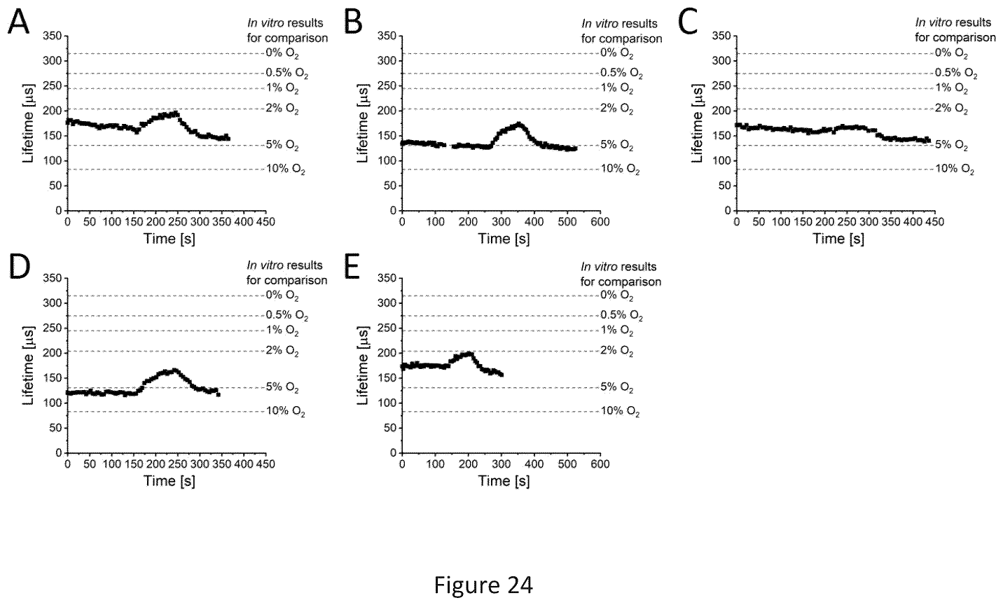

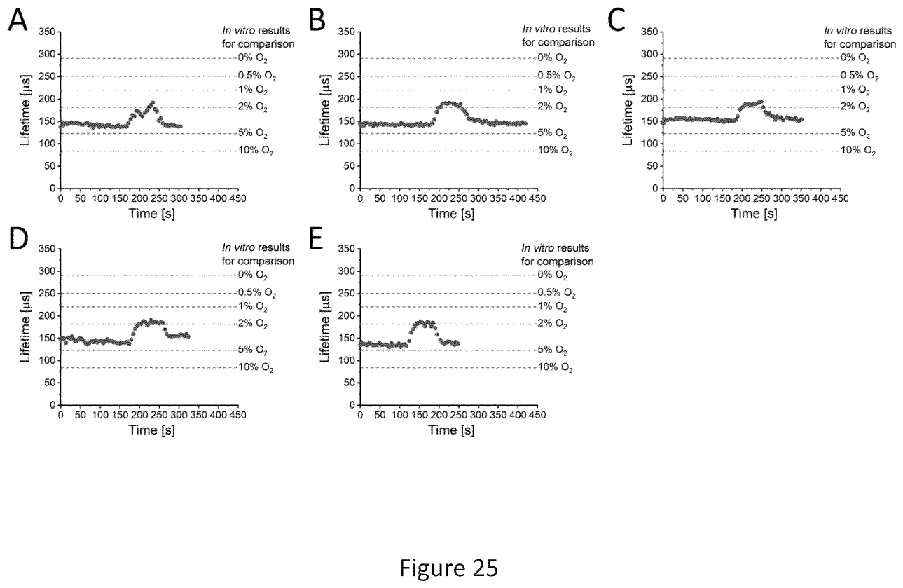

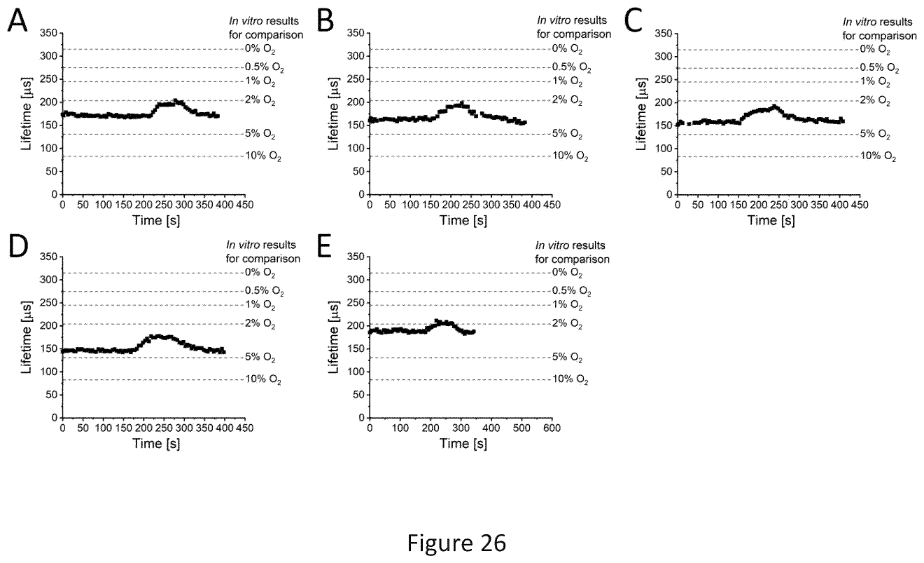

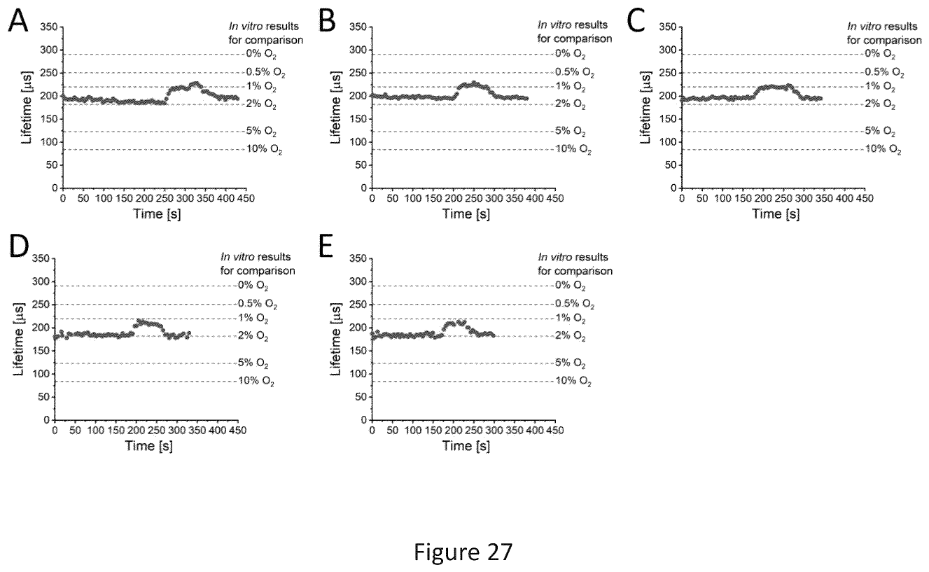

PdBMAP-containing silk sponges and 10 w/v % silk films were implanted in rats to facilitate a histological assessment and an evaluation of oxygen-sensing properties in vivo (FIGS. 21 and 22-27). For samples resected from the tissue at days 7, 21, and 28 (FIGS. 21B and 22-23), there was little inflammatory response in accordance with prior studies.[38] While films exhibited some remodeling at the interface with the tissue and sample, the sponge achieved successful tissue integration by day 28. Histological analysis showed cellular infiltration into the sponges with cell nuclei and extracellular matrix deposition throughout the sample. Over time, the implantation site border resembled healthy tissue as evidenced by the incorporation of vasculature, increased extracellular matrix deposition, and the decreased appearance of a defined border geometry from the sample.

In parallel with histological assessments, the oxygen-sensing performance was monitored from 14 to 28 days post-implantation in two different rats (FIGS. 21C-21D). On each assessment day, implanted sensors responded to changes in oxygen via a nose cone removal experiment (FIG. 21C and FIGS. 24-27). The initial region of these lifetime versus time plots appeared flat and corresponded to the rat inhaling a hyperoxic anesthesia/O₂ mixture via a nose cone. When the nose cone was removed for ˜60 s, the lifetime began increasing within seconds, indicating a lower tissue oxygenation while the rat breathed ambient air. When the nose cone was readministered, the lifetime decreased. In general, the rate of change for the sponges was greater than that observed for the films with the sponge composition lifetime being more likely to fully plateau before reapplication of the nose cone. FIG. 21D summarizes the results by displaying the average lifetimes when the rat was wearing or not wearing the nose cone (hyperoxia or normoxia, respectively). In cases when the response did not fully plateau after nose cone removal, the maximum lifetime was used instead. For each sponge sample, the observed lifetime for a given condition was fairly consistent over the course of 14 to 28 days.

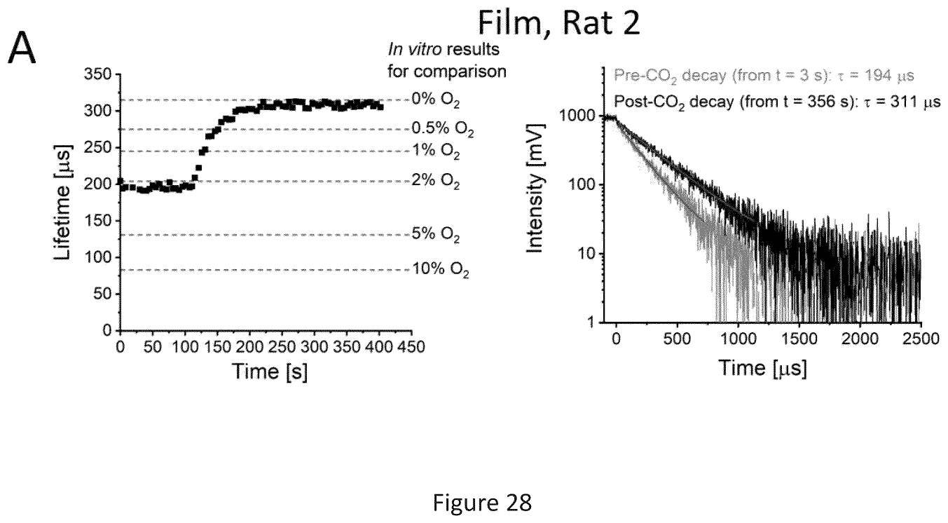

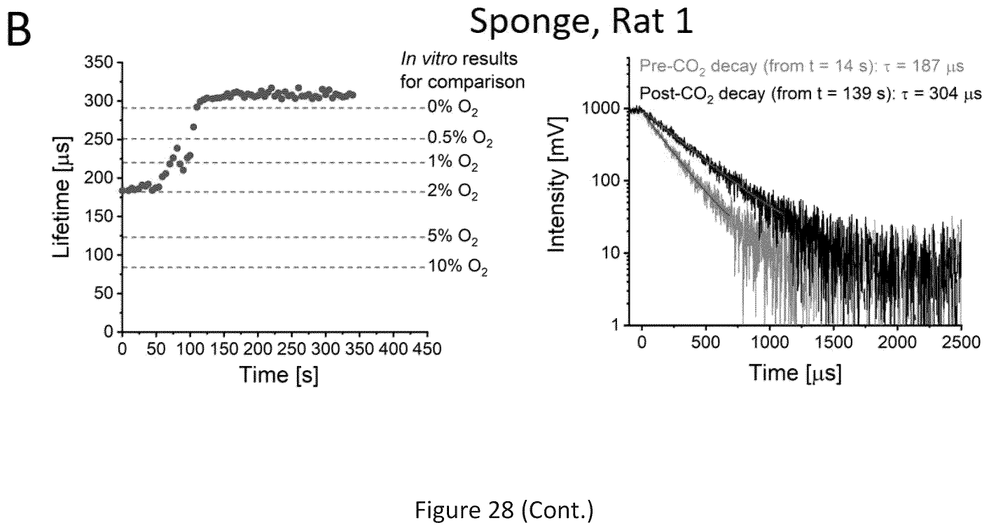

The final test of sensor functionality was performed on day 28 as the lifetime of the implanted compositions were monitored versus time during the standard CO₂ euthanasia process (FIG. 28). The rat was initially exposed to ambient oxygen levels before the chamber was flooded with CO₂. At this time, the lifetime increased within seconds and eventually plateaued at a value reminiscent of the in vitro deaerated lifetime. Note that the deviation that occurred between 85-100 s for the sponge sample corresponded to a real event, namely the rat briefly waking up. The rat was no longer awake by t=105 s, and the response no longer appeared to deviate significantly.

### Discussion

The ability to continuously monitor tissue oxygenation with an implantable, optical oxygen sensor[10,11,26] is desired for athletic monitoring[44] and the treatment of various diseases.[3-9] A bioresorbable implant could increase user acceptance, particularly for short-term applications (e.g., monitoring tissue oxygenation during an illness or hospital visit). Aqueous-based silk-PdBMAP oxygen-sensing silk films have been previously demonstrated, for which degradation time could be tuned without changing the chemical composition[38], an advantage compared to other bioresorbable, optical oxygen sensors consisting of synthetic polymers[16,29-32] or synthetic/natural polymer blends.[16,29] However, these silk sensors were only demonstrated in a non-porous format and exhibited clearly downward-curving Stern-Volmer plots, indicating chromophore environment heterogeneity. The current work demonstrates that HFIP-based processing can both improve chromophore incorporation/sensor interpretability and aid tissue integration via the introduction of controlled porosity. Additionally, evaluation via an in vivo rodent model demonstrated consistent performance over time, even during the initial degradation process.

Using HFIP-based processing, silk-PdBMAP compositions were successfully demonstrated in the form of non-porous films and porous sponges (FIG. 1). Since sponge fabrication involves the incorporation of water-soluble salts, the use of HFIP prevented dissolution of the non-soluble NaCl particles prior to the leaching step, allowing pore size to be directly related to the size of incorporated salt crystals. In this work, 100-450 μm particles were selected to achieve interconnected pores large enough to promote cellular infiltration and tissue integration. Indeed, the silk-PdBMAP sponges successfully improved tissue acceptance and integration as anticipated. The porous nature enabled cellular infiltration and matrix remodeling as represented by the purple and pink staining, respectively, in the histological analysis of explanted tissues (FIGS. 21B and 22-23). This sensor integration and tissue remodeling proceeded throughout the 28 day study with the extracellular space at the borders becoming indistinguishable from surrounding healthy tissue with regard to vascular structures. Although aqueous-based processing of porous silk sponges is possible, water-soluble NaCl salt crystals substantially dissolve during the process producing variable pore sizes. This process also involves leaving the aqueous silk solution covered for 24 hr[45], making the processing incompatible with the incorporation of a water-insoluble chromophore (e.g., PdBMAP) that aggregates under these conditions.

Although residual solvent could present biocompatibility issues, proper sample preparation avoids this concern. The HFIP-processed silk films and sponges offered excellent cytocompatibility as evidenced by the favorable histological assessment (FIGS. 21B and 22-23) as well as the live/dead assay and metabolic activity of human dermal fibroblasts seeded on the scaffolds (FIG. 5A-B). These results indicated that the fabrication methods employed were sufficient to render the materials biocompatible as residual solvent was removed when the samples were under vacuum. This observation aligns with other HFIP-based silk[46] and non-silk[47,48] materials due to the volatility of HFIP (boiling point: 59° C.).

Additionally, the use of an organic solvent for which the chromophore is readily soluble allowed for facile incorporation of the water-insoluble chromophore into the silk-PdBMAP composites. Compared to aqueous-processed materials, the homogeneity of the chromophore in the silk matrix was improved as evidenced by more monoexponential decays and increased Stern-Volmer plot linearity. While aqueous-based silk-PdBMAP films[38] exhibited clearly downward-curving Stern-Volmer plots, the HFIP-derived films exhibited linear Stern-Volmer plots, and the slightly downward-curving behavior of the silk-PdBMAP sponge was readily estimated with a linear fit (R²=0.997, FIG. 13). The ability to use a linear Stern-Volmer plot to describe or estimate sensor behavior simplifies calibration as it can be described by two parameters (τ₀ and K_{SV}) instead of four (τ₀, K_{SV,1}, K_{SV,2}, and f_{0,1}). One possible explanation for the apparent increase in chromophore heterogeneity for the sponge is the longer solvent evaporation time that resulted from the thicker sponge-based composition cast onto a bed of salt particles. This explanation aligns with the fact that rapid solvent evaporation generally helps preserve the solution state and impart chromophore environment homogeneity[47,49].

Furthermore, while aqueous-derived silk-PdBMAP films exhibited biexponential decays[38], the HFIP-derived materials exhibited approximately monoexponential decays (<5% O₂) or decays that could be accurately approximated with a monoexponential fit (≥5% O₂). Increased biexponential character at higher oxygen concentrations indicates that this deviation from monoexponential character may arise from varied oxygen permeability in different chromophore environments. Such an effect would not exist in the absence of oxygen and would exert a greater effect as dissolved oxygen concentration increases. Although HFIP-derived materials exhibited notable biexponential character ≥5% O₂, this may only be prevalent at higher dissolved oxygen concentrations than typically anticipated in vivo and could be accurately estimated with a monoexponential decay, preserving their utility for real-time continuous monitoring. For example, for 10 w/v % silk films at 5 and 10% O₂, τₘ (biexponential fit) and τ (monoexponential fit) were essentially identical (<0.2% difference) but differed by up to 3.4% for the aqueous-based equivalent.[38] A heterogeneous chromophore environment could result from either variable oxygen permeability and/or to values in different areas of the matrix (i.e., hydrophobic versus hydrophilic silk domains) and/or chromophore aggregation. However, aggregation likely played at least a partial role in previous aqueous-processed films,[38] since the chromophore environment homogeneity was improved in the HFIP-based films despite both being comprised of the same silk material.

Therefore, the more monoexponential decay curves and linear Stern-Volmer plots of HFIP-based silk-PdBMAP sensors improve interpretability for continuous real-time monitoring. Most importantly, a method with more homogeneous chromophore incorporation allows for more consistent photophysical properties. When chromophore aggregation is present, small changes in the processing parameters or environmental conditions during fabrication could impact the extent of aggregation and, therefore, the resultant photophysical properties. For example, careful control over the film processing parameters and environmental conditions (e.g., 80-90% humidity during initial film drying stage) was necessary to avoid overt aggregation with our previous work that incorporated water-insoluble PdBMAP into silk films using DI H₂O+<1% DMSO.[38]

Both silk-PdBMAP films and sponges exhibited a high maximum (i.e., deaerated) lifetime (τ₀=˜300 μs) and similar sensitivities (K_{SV}: ˜2.7-3.2×10⁴ M⁻¹). One parameter to assess where a sensor functions best is 1/K_{SV} (in the case of a linear Stern-Volmer plot) or where the lifetime is quenched to 50% of its maximum.[15] In all cases, this value was ˜3.4-3.5% O₂ (˜31-32 μM) which falls within the expected region of healthy physiological levels.[50] Therefore, these materials should be well-suited to monitor typical tissue oxygenation levels and to detect deviations from normal behavior including hyperoxia. The observed Stern-Volmer quenching rate constant was ˜3.2-4.6× lower compared to PdBMAP-containing PCL and PCL: gelatin electrospun nanofiber sensors.[16] Although these other formulations are advantageous for detecting hypoxia, the more moderate sensitivity of the silk-PdBMAP composites offers an improved dynamic range. Note that while the choice of chromophore arguably exerts the greatest impact on sensitivity, the matrix material can significantly affect T₀[29], and k_{q} increases with oxygen diffusivity.[51] Additionally, the sensitivity was similar to that observed for aqueous-processed silk-PdBMAP films[38], but as aforementioned, the HFIP-based processing improved chromophore environment homogeneity, thereby increasing interpretability and ease of processing.

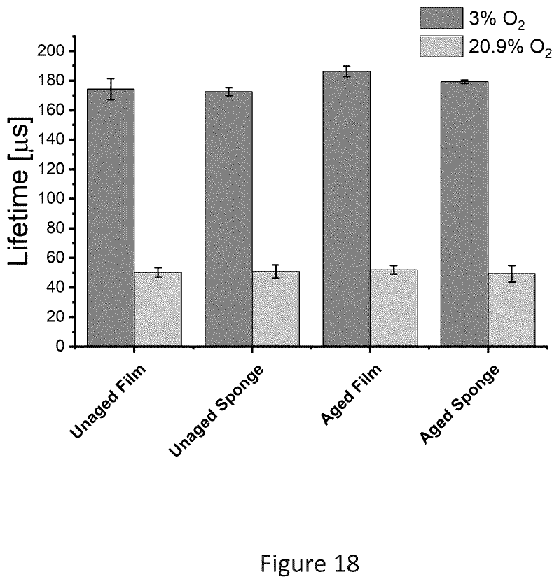

The similar oxygen-sensitivity of all HFIP-derived silk-PdBMAP compositions (Table 1) indicates that degradation time can be varied without significantly impacting photophysical properties. For films, the observed faster degradation of thinner samples is logical considering that the enzymatic degradation occurs at the surface of the film[33,52]. This mechanism also explains why the highly porous sponge, which has a higher surface area, degraded faster than the films. Overall, the degradation time can be tuned by adjusting the silk film or sponge structure without appreciable changes in photophysical properties/sensing capabilities. Note that degradation occurred faster with protease XIV than α-chymotrypsin as expected, since the former can readily degrade the crystalline β-sheet regions, while the latter primarily attacks amorphous regions.[52-54] While protease XIV offers a means to achieve more accelerated degradation, the mechanism of α-chymotrypsin is more representative of the behavior in vivo.[54] Interestingly, minimal changes to photophysical properties were observed to either the silk-PdBMAP sponge or 10 w/v % silk-PdBMAP film after a 4-day exposure to α-chymotrypsin (FIGS. 18-20), indicating good robustness for use in vivo.

In fact, PdBMAP-containing silk films and sponges successfully monitored a rat under normoxia and hyperoxia during nose cone removal experiments from days 14 to 28 (FIGS. 21C-D and 24-27) and also detected a change from normoxia to hypoxia via the exposure to CO₂ during euthanasia (FIG. 28). To our knowledge, this is the first published example of monitoring a bioresorbable, optical oxygen sensor in vivo over the course of multiple days and is an important stride toward furthering the readiness of such technology for clinical point-of-care use. In all cases, responses to external stimuli were rapid, and the lifetimes follow the expected trend of being increasingly quenched with an increase in dissolved oxygen concentrations (lifetime: hypoxia (CO₂)>normoxia (no nose cone)>hyperoxia (nose cone)). Although k_{q} and/or to may be altered to some degree in vivo, the results compare reasonably well with expected physiological tissue oxygen concentrations. For example, the lifetime after exposure to 100% CO₂ was within 6% of the in vitro to. Interestingly, tissue oxygenation appears to be consistently greater in rat 1 than in rat 2 and this observation may be related to the large size difference between the two animals (rats 1 and 2 weighed 182 and 368 g, respectively). This difference highlights the utility of these implantable sensors to study changes in tissue oxygenation on an individual basis which could be critical to their use in studying and/or treating various diseases.

Although both the film and sponge retained functionality over the 28-day study, the porous sponge exhibited a greater rate of change in response to physical stimuli (e.g., nose cone removal and introduction of CO₂), and there was a greater likelihood for a plateaued lifetime value to be reached during the 60 s nose cone removal experiments (FIGS. 21C and 24-27). The high surface area and successful tissue integration of the porous sponge likely resulted in its more rapid response time and contributed to its more consistent performance over time (FIG. 21D). These results are supported by the histological analysis affirming that the microenvironment surrounding the sponge is reminiscent of actual tissue (FIG. 21B). The presence of vascular structures within the pores suggests improved diffusion and oxygen transfer within the construct, leading to more rapid and accurate sensing with outcomes that are representative of the healthy, uninterrupted interstitial milieu.

### CONCLUSIONS

Overall, this work successfully demonstrates a strategy to achieve tissue-integrating, bioresorbable optical oxygen sensors and highlights the advantages conferred by HFIP-based processing, namely increased chromophore environment homogeneity/interpretability and the ability to achieve tissue-integrating scaffolds. For both non-porous silk films and porous sponges, oxygen-sensitivity was imparted through incorporation of a phosphorescent Pd (II) benzoporphyrin chromophore, the lifetime of which is dynamically quenched by dissolved oxygen. The oxygen-sensitivity of the fabricated composites was similar, offering a means to tune degradation time without appreciably changing sensor properties. In addition, the moderate oxygen-sensitivity of these compositions offers a dynamic range ideal for monitoring physiological oxygen concentrations. Although both silk-PdBMAP films and sponges functioned well in vivo, the sponge achieved successful tissue integration, exhibited more consistent performance over time, and responded more quickly in response to external stimuli. This work makes significant strides in advancing the readiness of this technology that could serve as the basis for optical detection of other physiological analytes in the future.

### REFERENCES

[1] A. Ahmad Tarar, U. Mohammad, S. K. Srivastava, Wearable skin sensors and their challenges: A review of transdermal, optical, and mechanical sensors, Biosensors. 10 (2020) 56. https://doi.org/10.3390/BIOS10060056.
[2] H. C. Koydemir, A. Ozcan, Wearable and implantable sensors for biomedical applications, Annu. Rev. Anal. Chem. 11 (2018) 127-146. https://doi.org/10.1146/annurev-anchem-061417-125956.
[3] V. De Santis, M. Singer, Tissue oxygen tension monitoring of organ perfusion: Rationale, methodologies, and literature review, Br. J. Anaesth. 115 (2015) 357-365. https://doi.org/10.1093/bja/aev162.
[4] V. Mirabello, F. Cortezon-Tamarit, S. I. Pascu, Oxygen-sensing, hypoxia tracing and in vivo imaging with functional metalloprobes for the early detection of non-communicable diseases, Front. Chem. 6 (2018) 27. https://doi.org/10.3389/fchem.2018.00027.
[5] M. Montero-Baker, A. Schwindt, T. Bisdas, Potential utility of tissue oxygen sensors in perfusion monitoring, Endovasc. Today. 17 (2018) 60-64.
[6] I. G. Lempesis, R. L. J. van Meijel, K. N. Manolopoulos, G. H. Goossens, Oxygenation of adipose tissue: A human perspective, Acta Physiol. 228 (2020) e13298. https://doi.org/10.1111/apha.13298.
[7] D. Goldman, R. M. Bateman, C. G. Ellis, Effect of sepsis on skeletal muscle oxygen consumption and tissue oxygenation: Interpreting capillary oxygen transport data using a mathematical model, Am. J. Physiol.-Hear. Circ. Physiol. 287 (2004) H2535-H2544. https://doi.org/10.1152/ajpheart.00889.2003.
[8] A. Borghi-Silva, C. C. Oliveira, C. Carrascosa, J. Maia, D. C. Berton, F. Queiroga, E. M. Ferreira, D. R. Almeida, L. E. Nery, J. A. Neder, Respiratory muscle unloading improves leg muscle oxygenation during exercise in patients with COPD, Thorax. 63 (2008) 910-915. https://doi.org/10.1136/thx.2007.090167.
[9] R. J. M. Cajanding, Silent hypoxia in COVID-19 pneumonia: State of knowledge, pathophysiology, mechanisms, and management, AACN Adv. Crit. Care. 33 (2022) 143-153. https://doi.org/10.4037/aacnacc2022448.
[10] J. S. Chien, M. Mohammed, H. Eldik, M. M. Ibrahim, J. Martinez, S. P. Nichols, N. Wisniewski, B. Klitzman, Injectable phosphorescence-based oxygen biosensors identify post ischemic reactive hyperoxia, Sci. Rep. 7 (2017) 1-10. https://doi.org/10.1038/s41598-017-08490-0.
[11] S. P. Nichols, M. K. Balaconis, R. M. Gant, K. Y. Au-Yeung, N. A. Wisniewski, Long-term in vivo oxygen sensors for peripheral artery disease monitoring, Adv. Exp. Med. Biol. 1072 (2018) 351-356. https://doi.org/10.1007/978-3-319-91287-5_56.
[12] J.-A. Collins, A. Rudenski, J. Gibson, L. Howard, R. O'Driscoll, Relating oxygen partial pressure, saturation and content: The haemoglobin-oxygen dissociation curve, Breathe. 11 (2015) 194-201. https://doi.org/10.1183/20734735.001415.
[13] B. A. DeGraff, J. N. Demas, Luminescence-based oxygen sensors, in: C. D. Geddes, J. R. Lakowicz (Eds.), Rev. Fluoresc. 2005, Springer, Boston, MA, 2005: pp. 125-151. https://doi.org/10.1007/0-387-23690-2_6.
[14] X. Wang, H. Chen, Y. Zhao, X. Chen, X. Wang, Optical oxygen sensors move towards colorimetric determination, TrAC Trends Anal. Chem. 29 (2010) 319-338. https://doi.org/10.1016/j.trac.2010.01.004.
[15] A. Mills, Controlling the sensitivity of optical oxygen sensors, Sensors Actuators, B Chem. 51 (1998) 60-68. https://doi.org/10.1016/S0925-4005 (98) 00211-1.
[16] K. F. Presley, B. M. Reinsch, D. B. Cybyk, J. T. Ly, R. M. Schweller, M. J. Dalton, J. J. Lannutti, T. A. Grusenmeyer, Oxygen-sensing performance of biodegradable electrospun nanofibers: Influence of fiber composition and core-shell geometry, Sensors Actuators, B Chem. 329 (2021) 129191. https://doi.org/10.1016/j.snb.2020.129191.
[17] E. R. Carraway, J. N. Demas, B. A. DeGraff, Luminescence quenching mechanism for microheterogeneous systems, Anal. Chem. 63 (1991) 332-336. https://doi.org/10.1021/ac00004a006.
[18] J. N. Demas, B. A. DeGraff, W. Xu, Modeling of luminescence quenching-based sensors: comparison of multisite and nonlinear gas solubility models, Anal. Chem. 67 (1995) 1377-1380. https://doi.org/10.1021/ac00104a012.
[19] P. Douglas, K. Eaton, Response characteristics of thin film oxygen sensors, Pt and Pd octaethylporphyrins in polymer films, Sensors Actuators B Chem. 82 (2002) 200-208. https://doi.org/10.1016/S0925-4005 (01) 01006-1.
[20] A. Mills, Optical sensors for oxygen: A log-gaussian multisite-quenching model, Sensors Actuators B Chem. 51 (1998) 69-76. https://doi.org/10.1016/S0925-4005 (98) 00221-4.
[21] A. Mills, Response characteristics of optical sensors for oxygen: A model based on a distribution in τ0 or kq, Analyst. 124 (1999) 1301-1307. https://doi.org/10.1039/A902153A.
[22] A. Mills, Response characteristics of optical sensors for oxygen: A model based on a distribution in τ0 and kq, Analyst. 124 (1999) 1309-1314. https://doi.org/10.1039/a902155h.
[23] X. Wang, O. S. Wolfbeis, Optical methods for sensing and imaging oxygen: Materials, spectroscopies and applications, Chem. Soc. Rev. 43 (2014) 3666-3761. https://doi.org/10.1039/c4cs00039k.
[24] C. Li, C. Guo, V. Fitzpatrick, A. Ibrahim, M. J. Zwierstra, P. Hanna, A. Lechtig, A. Nazarian, S. J. Lin, D. L. Kaplan, Design of biodegradable, implantable devices towards clinical translation, Nat. Rev. Mater. 5 (2020) 61-81. https://doi.org/10.1038/s41578-019-0150-z.
[25] R. R. Anderson, J. A. Parrish, The optics of human skin, J. Invest. Dermatol. 77 (1981) 13-19. https://doi.org/10.1111/1523-1747.ep12479191.
[26] A. N. Bashkatov, E. A. Genina, V. I. Kochubey, V. V. Tuchin, Optical properties of human skin, subcutaneous and mucous tissues in the wavelength range from 400 to 2000 nm, J. Phys. D. Appl. Phys. 38 (2005) 2543-2555. https://doi.org/10.1088/0022-3727/38/15/004.
[27] J. E. Rogers, K. A. Nguyen, D. C. Hufnagle, D. G. McLean, W. Su, K. M. Gossett, A. R. Burke, S. A. Vinogradov, R. Pachter, P. A. Fleitz, Observation and Interpretation of Annulated Porphyrins: Studies on the Photophysical Properties of meso-Tetraphenylmetalloporphyrins, J. Phys. Chem. A. 107 (2003) 11331-11339. https://doi.org/10.1021/jp0354705.
[28] M. F. Montero-Baker, K. Y. Au-Yeung, N. A. Wisniewski, S. Gamsey, L. Morelli-Alvarez, J. L. Mills, M. Campos, K. L. Helton, The first-in-man “si se puede” Study for the use of micro-oxygen sensors (MOXYs) to determine dynamic relative oxygen indices in the feet of patients with limb-threatening ischemia during endovascular therapy, J. Vasc. Surg. 61 (2015) 1501-1510.el. https://doi.org/10.1016/j.jvs.2014.12.060.
[29] D. B. Cybyk, B. M. Reinsch, K. F. Presley, R. M. Schweller, A. Tedeschi, T. Burns, F. J. Chaparro, J. T. Ly, M. J. Dalton, M. Lozano, T. A. Grusenmeyer, J. J. Lannutti, Biodegradable oxygen biosensors via electrospinning, Med. Devices Sensors. 4 (2021) e¹⁰¹⁴⁹. https://doi.org/10.1002/mds3.10149.
[30] C. A. DeRosa, S. A. Seaman, A. S. Mathew, C. M. Gorick, Z. Fan, J. N. Demas, S. M. Peirce, C. L. Fraser, Oxygen-sensing difluoroboron β-diketonate polylactide materials with tunable dynamic ranges for wound imaging, ACS Sensors. 1 (2016) 1366-1373. https://doi.org/10.1021/acssensors.6b00533.
[31] D. T., Bowers, M. L. Tanes, A. Das, Y. Lin, N. A. Keane, R. A. Neal, M. E. Ogle, K. L. Brayman, C. L. Fraser, E. A. Botchwey, Spatiotemporal oxygen-sensing using dual emissive boron dye-polylactide nanofibers, ACS Nano. 8 (2014) 12080-12091. https://doi.org/10.1021/nn504332j.
[32] C. A. DeRosa, J. Samonina-Kosicka, Z. Fan, H. C. Hendargo, D. H. Weitzel, G. M. Palmer, C. L. Fraser, Oxygen-sensing difluoroboron dinaphthoylmethane polylactide, Macromolecules. 48 (2015) 2967-2977. https://doi.org/10.1021/acs.macromol.5b00394.
[33] Y. Cao, B. Wang, Biodegradation of silk biomaterials, Int. J. Mol. Sci. 10 (2009) 1514-1524. https://doi.org/10.3390/ijms10041514.
[34] D. N. Rockwood, R. C. Preda, T. Yücel, X. Wang, M. L. Lovett, D. L. Kaplan, Materials fabrication from Bombyx mori silk fibroin, Nat. Protoc. 6 (2011) 1612-1631. https://doi.org/10.1038/nprot.2011.379.
[35] B. Kundu, R. Rajkhowa, S. C. Kundu, X. Wang, Silk fibroin biomaterials for tissue regenerations, Adv. Drug Deliv. Rev. 65 (2013) 457-470. https://doi.org/10.1016/j.addr.2012.09.043.
[36] A. B. Li, J. A. Kluge, N. A. Guziewicz, F. G. Omenetto, D. L. Kaplan, Silk-based stabilization of biomacromolecules, J. Control. Release. 219 (2015) 416-430. https://doi.org/10.1016/j.jconrel.2015.09.037.
[37] K. Numata, D. L. Kaplan, Silk-based delivery systems of bioactive molecules, Adv. Drug Deliv. Rev. 62 (2010) 1497-1508. https://doi.org/10.1016/j.addr.2010.03.009.
[38] T. Falcucci, K. F. Presley, J. Choi, V. Fizpatrick, J. Barry, J. K. Sahoo, J. T. Ly, T. A. Grusenmeyer, M. J. Dalton, D. L. Kaplan, Degradable silk-based subcutaneous oxygen sensors, Adv. Funct. Mater. 32 (2022) 2202020. https://doi.org/10.1002/adfm.202202020.
[39] X. Hu, K. Shmelev, L. Sun, E.-S. Gil, S.-H. Park, P. Cebe, D. L. Kaplan, Regulation of silk material structure by temperature-controlled water vapor annealing, Biomacromolecules. 12 (2011) 1686-1696. https://doi.org/10.1021/bm200062a.
[40] R. Nazarov, H. J. Jin, D. L. Kaplan, Porous 3-D scaffolds from regenerated silk fibroin, Biomacromolecules. 5 (2004) 718-726. https://doi.org/10.1021/bm034327e.
[41] K. F. Presley, J. T. Ly, J. R. Deneault, M. J. Dalton, T. A. Grusenmeyer, Time-resolved spectroscopy of phosphorescent oxygen sensors in a relevant in vitro environment for biomedical applications, Edinburgh Instruments Appl. Notes. (n.d.). https://www.edinst.com/us/time-resolved-spectroscopy-of-phosphorescent-oxygen sensors-in-a-relevant-in-vitro-environment-for-biomedical-applications/.
[42] 4.2.2.18 Fitting with Convolution, OriginLab. (n.d.). https://www.originlab.com/doc/Tutorials/Fitting-Convolution.
[43] A. Sillen, Y. Engelborghs, The Correct Use of “Average” Fluorescence Parameters, Photochem. Photobiol. 67 (1998) 475-486. https://doi.org/10.1111/j.1751-1097.1998.tb09082.x.
[44] K. J. Smith, F. Billaut, Tissue oxygenation in men and women during repeated-sprint exercise, Int. J. Sports Physiol. Perform. 7 (2012) 59-67. https://doi.org/10.1123/ijspp.7.1.59.
[45] U.-J. Kim, J. Park, H. J. Kim, M. Wada, D. L. Kaplan, Three-dimensional aqueous-derived biomaterial scaffolds from silk fibroin, Biomaterials. 26 (2005) 2775-2785. https://doi.org/10.1016/j.biomaterials.2004.07.044.
[46] S. E. L. Vidal, K. A. Tamamoto, H. Nguyen, R. D. Abbott, D. M. Cairns, D. L. Kaplan, 3D biomaterial matrix to support long term, full thickness, immuno-competent human skin equivalents with nervous system components, Biomaterials. 198 (2019) 194-203. https://doi.org/10.1016/j.biomaterials.2018.04.044.
[47] R. Xue, P. Behera, M. S. Viapiano, J. J. Lannutti, Rapid response oxygen-sensing nanofibers, Mater. Sci. Eng. C. 33 (2013) 3450-3457. https://doi.org/10.1016/j.msec.2013.04.030.
[48] J. Lee, G. Tae, Y. H. Kim, I. S. Park, S.-H. Kim, S. H. Kim, The effect of gelatin incorporation into electrospun poly(l-lactide-co-ε-caprolactone) fibers on mechanical properties and cytocompatibility, Biomaterials. 29 (2008) 1872-1879. https://doi.org/10.1016/j.biomaterials.2007.12.029.
[49] K. Presley, M. Shahhosseini, D. Shi, C. Castro, J. Lannutti, Analysis of long-term optical performance of phosphorescent oxygen-sensing polymeric nanofibers, Polym. Test. 80 (2019) 106127. https://doi.org/10.1016/j.polymertesting.2019.106127.
[50] A. Carreau, B. El Hafny-Rahbi, A. Matejuk, C. Grillon, C. Kieda, Why is the partial oxygen pressure of human tissues a crucial parameter? Small molecules and hypoxia, J. Cell. Mol. Med. 15 (2011) 1239-1253. https://doi.org/10.1111/j.1582-4934.2011.01258.x.
[51] S. M. Borisov, Fundamentals of Quenched Phosphorescence 02 Sensing and Rational Design of Sensor Materials, in: D. B., Papkovsky, R. I., Dmitriev (Eds.), Quenched-Phosphorescence Detect. Mol. Oxyg. Appl. Life Sci., The Royal Society of Chemistry, London, U K, 2018: pp. 1-18. http://dx.doi.org/10.1039/9781788011754-00001.
[52] T. Arai, G. Freddi, R. Innocenti, M. Tsukada, Biodegradation of Bombyx mori silk fibroin fibers and,films, J. Appl. Polym. Sci. 91 (2004) 2383-2390. https://doi.org/10.1002/app.13393.
[53] M. Li, M. Ogiso, N. Minoura, Enzymatic degradation behavior of porous silk fibroin sheets, Biomaterials. 24 (2003) 357-365. https://doi.org/10.1016/S0142-9612 (02) 00326-5.
[54] K. Numata, P. Cebe, D. L. Kaplan, Mechanism of enzymatic degradation of beta-sheet crystals, Biomaterials. 31 (2010) 2926-2933. https://doi.org/10.1016/j.biomaterials.2009.12.026.

### Miscellaneous

Unless otherwise specified or indicated by context, the terms “a”, “an”, and “the” mean “one or more.” For example, “a molecule” should be interpreted to mean “one or more molecules.”

As used herein, the terms “substantially zero” or “substantially 0%” refer to a value that is lower than a detection limit, lower than a noise value, or otherwise so small as to be considered insignificant to a person having ordinary skill in the art.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An oxygen-sensing material comprising:
a plurality of oxygen-sensing chromophores embedded in a solid matrix comprising silk fibroin in an amount by weight of at least 50% of a total weight of the solid matrix,
wherein the solid matrix is at least partially optically transparent to light at one or more wavelengths between 600 nm and 1300 nm,
wherein the solid matrix is biocompatible and biodegradable,
wherein the solid matrix is not a hydrogel,
wherein the plurality of oxygen-sensing chromophores are distributed throughout the solid matrix.

2. A method of making an oxygen-sensing material, the method comprising:
a) dissolving silk fibroin and a plurality of oxygen-sensing chromophores into an organic solvent;
b) removing the organic solvent, thereby resulting in the oxygen-sensing material comprising the plurality of oxygen-sensing chromophores embedded in a solid matrix comprising the silk fibroin,
wherein an amount of silk fibroin dissolved in step a) is selected to provide the solid matrix with silk fibroin in an amount by weight of at least 50% of a total weight of the solid matrix, wherein the removing is tailored to provide the following properties:
the solid matrix is at least partially optically transparent to light at one or more wavelengths between 600 nm and 1300 nm;
the solid matrix is biocompatible and biodegradable;
the solid matrix is not a hydrogel; and
and the plurality of oxygen-sensing chromophores are distributed throughout the solid matrix.

3. The oxygen-sensing material of claim 1, wherein the plurality of oxygen-sensing chromophores comprise Pt(II) benzoporphyrins, Pd(II) benzoporphyrins, or a combination thereof.

4. The oxygen-sensing material of claim 1, wherein the plurality of oxygen-sensing chromophores comprise Pd(II) benzoporphyrins.

5. The oxygen-sensing material of claim 1, wherein the plurality of oxygen-sensing chromophores comprise Pd(II) tetramethacrylated benzoporphyrins.

6. The oxygen-sensing material of claim 1, wherein the plurality of oxygen-sensing chromophores are present in the solid matrix in an amount by weight of between 0.05% and 0.25% of the total weight of the solid matrix.

7. The oxygen-sensing material of claim 1, wherein the solid matrix comprises silk fibroin in an amount by weight of at least 55% of the total weight of the solid matrix.

8. The oxygen-sensing material of claim 1, wherein the plurality of oxygen-sensing chromophores have a solubility in water at pH 7.4 and 37° C. of less than 0.01 mg/mL

9. The oxygen-sensing material of claim 1, wherein the oxygen-sensing material has a compressive modulus of at least 1 kPa.

10. The oxygen-sensing material of claim 1, wherein the oxygen-sensing material has a tensile modulus of at least 1 MPa.

11. The oxygen-sensing material of claim 1, wherein the oxygen-sensing material has a deaerated phosphorescence lifetime as measured at substantially zero soluble oxygen concentration, wherein the oxygen-sensing material provides a phosphorescence lifetime that is 50% of the deaerated phosphorescence lifetime at a soluble oxygen concentration of between 5 μM and 50 μM.

12. The oxygen-sensing material of claim 1, wherein the oxygen-sensing material has a dissolution rate in pure water at 37° C. of at least 70% of initial mass remains after 2 weeks.

13. The oxygen-sensing material of claim 1, wherein the solid matrix is at least partially enzymatically degradable.

14. The oxygen-sensing material of claim 1, wherein the solid matrix is porous.

15. The oxygen-sensing material of claim 1, wherein the solid matrix is a foam.

16. The oxygen-sensing material of claim 1, wherein the solid matrix is a film.

17. The oxygen-sensing material of claim 1, wherein the solid matrix consists essentially of the silk fibroin.

18. The oxygen-sensing material of claim 1, wherein at least 50% of the silk fibroin has a molecular weight of at least 60 kDa.

19. The oxygen-sensing material of claim 1, wherein the silk fibroin has a number average molecular weight of at least 80 kDa and the number average molecular weight is at most 400 kDa.

20. The oxygen-sensing material of claim 1, wherein a phosphorescence decay curve measured from the oxygen-sensing material has a biexponential fit resulting in an amplitude average lifetime that varies from the phosphorescence lifetime resulting from a monoexponential fit of the phosphorescence decay curve by at most 10%.

21. A method of using an oxygen-sensing material, the method comprising:
acquiring phosphorescence data from an implantable oxygen sensor material,
wherein the implantable oxygen sensor material comprises a plurality of oxygen-sensing chromophores embedded in and distributed throughout a solid matrix comprising silk fibroin in an amount by weight of at least 50% of a total weight of the solid matrix, wherein the solid matrix is at least partially optically transparent to light at one or more wavelengths between 600 nm and 1300 nm,
wherein the solid matrix is biocompatible and biodegradable, and
wherein the solid matrix is not a hydrogel.

22. The method of claim 21, the method further comprising: extracting a reading that is indicative of a tissue oxygenation level for a subject from the phosphorescence data.
